(19) United States
(12) Patent Application Publication
(10) Pub. No.: US 2023/0092775 A1
(43) Pub. Date: Mar. 23, 2023

(54) DELIVERY AND DEPLOYMENT OF A PROSTATIC IMPLANT

(71) Applicant: Prodeon Medical Corporation, Taipei (TW)

(72) Inventors: Chun-Chia Juan, Taipei (TW); Zong-Lin Li, Taipei (TW); De-Yu Chu, Taipei (TW); Yu-Chen Lin, Taipei (TW); Chiu-Ming Hsu, Taipei (TW); Yu-Shih Weng, Taipei (TW); Ying-Siao Chen, Taipei (TW)

(21) Appl. No.: 17/948,489

(22) Filed: Sep. 20, 2022

Related U.S. Application Data
(60) Provisional application No. 63/246,040, filed on Sep. 20, 2021.

(51) Int. Cl. A61B 1/307; A61F 2/95; A61M 27/00

(52) U.S. Cl. CPC A61B 1/307; A61F 2/9517; A61M 27/008; A61B 2017/00274

(57) ABSTRACT

Devices and methods are disclosed for managing and/or treating body tissues obstructing a hollow body lumen, including the prostatic lobe tissues obstructing the urethra, for example conditions including benign prostatic hyperplasia (BPH), bladder outlet obstruction (BOO), benign prostatic obstruction (BPO) and associated lower urinary tract symptoms (LUTS). A prostatic implant deployment and delivery system may have a controlled release mechanism, a handle mechanism and an irrigation system. The controlled release mechanism includes a disengageable connection between a pusher member and the prostatic implant with a control member. The handle mechanism includes a plunger for advancing the pusher member and an actuator for withdrawing the control member. The irrigation system may define two fluid paths.

### RELATED APPLICATIONS

This application claims priority to U.S. Provisional Application No. 63/246,040, filed Sep. 20, 2021. The priority of this application is expressly claimed, and the disclosure is hereby incorporated by reference in its entirety.

### FIELD OF THE PRESENT DISCLOSURE

This disclosure relates to devices for managing or treating body tissues obstructing a hollow body lumen, such as the prostatic lobe tissues obstructing the urethra.

### BACKGROUND

The prostate is a walnut-shaped gland that wraps around the urethra through which urine is expelled from the bladder and plays a crucial role in the reproductive system of men. Although the gland starts out small, it tends to enlarge as a man ages. An excessively enlarged prostate results in a disease known as benign prostatic hyperplasia (BPH). Benign prostatic hyperplasia (BPH) refers to the abnormal, but non-malignant (non-cancerous) growth of the prostate observed very commonly in aging men. BPH is a chronic condition and is associated with the development of urinary outflow obstruction or luminal narrowing in the prostatic urethra. Bladder outlet obstruction (BOO) refers to a blockage at the base of the bladder that reduces or stops the flow of urine into the urethra and may be secondary to BPH. A range of related disorders referred to collectively as Lower Urinary Tract Symptoms (LUTS) can result, including sexual dysfunction, frequent urination, difficulty in voiding urine, urinary retention, urinary leakage, and urinary tract and bladder infections that worsen as the abnormal growth in the prostate enlarges and progresses.

Surgical procedures provide BPH relief by removing a significant portion the prostate tissue. Several traditional surgical procedures are available, all of which require hospitalization and some form of spinal, epidural, or general anesthesia. Transurethral resection of the prostate (TURP) is the main surgical treatment for BPH and remains the gold standard against which other treatments are compared. Traditional surgical techniques differ in the location of the incision made by the surgeon to access the prostate and in the method by which prostatic tissue is removed. For example, some surgeries use laser energy, heat, or radio frequency to remove tissue from the prostate. They include laser enucleation, photoselective vaporization (PVP), transurethral needle ablation (TUNA) using radiofrequency energy, transurethral microwave thermotherapy (TUMT) and transurethral incision of prostate (TUIP). However, these traditional surgical approaches to the treatment of BPH are invasive, non-reversible, and have significant drawbacks including the placement of a temporary catheter for a few months, risk of infection, loss of sexual function, urinary incontinence, and restenosis—wherein recurring hyperplasia of cells in the prostate regrow to cause a recurrence of the narrowing of the urethra opening and also a recurrence of the LUTS symptoms described above.

Although removing prostatic tissue relieves some BPH symptoms, tissue removal by traditional surgical approaches is irreversible and any adverse effects of the surgery may afflict the patient for life or affect the patients' quality of life. Moreover, surgical approaches are associated with the inherent risks from the surgery itself, risk of recurrence from the regrowth of removed prostatic tissue, and, depending on the extent of the disease and the particular surgical approach necessary for an individual patient, can require recovery periods as long as 3 to 6 weeks.

Because of the recognized drawbacks of traditional surgery, less invasive therapies have been developed and, depending on the extent of disease, may be chosen by patients and their physicians as an alternative to lifelong medication or surgery. These less invasive therapies may be suited for those patients not willing or medically not fit to have a surgical procedure performed under general anesthesia. In addition, younger patients also prefer a less invasive, reversible treatment without compromising sexual function, and leave the option of receiving a permanent, non-reversible treatment affecting sexual function at a later age. Further, since less invasive therapies permit treatment in the office or clinic using a local anesthetic, benefits include patient's comfort and healthcare system economy as compared to treatments under general anesthesia in a hospital setting.

Less invasive techniques include transurethral methods that actually remove enlarged prostatic tissue that are generally less traumatic than traditional surgery, but each destroys prostatic tissue and is irreversible. To avoid destroying the prostatic tissue, other therapeutic procedures have been developed that are designed to enlarge the diameter of the prostatic urethra without actual removal of tissue from the prostate gland, such as by implanting a device within the prostatic urethra that is designed to enlarge the diameter of the urethra. A prostatic implant involves a procedure wherein the urologist inserts a small device within the prostatic urethra which is narrowed by enlarged prostatic tissue. Once in place, the implant is designed to expand and help keep the urethra open by pushing out the tissue lobes, while preventing enlarged prostate tissue from total impingement and opening of the urethra. Ideally, prostatic implants eliminate the need to surgically remove prostatic tissue and are expected to reduce the risks of infection, sexual dysfunction, and incontinence, inherent and traditional to even less-invasive, surgical approaches. The procedure may also be designed to be reversible since the implants may be removed and additional surgical treatments may be performed in the future.

Accordingly, there is a need for an implant delivery and deployment system for the physician to be able to perform the procedure in an office using standard cystoscopes and common urological techniques used to examine the extent of BPH and obstruction in the prostatic urethra. It would be desirable for the delivery system to release the implant after confirming that it is placed in the correct position. Beneficial features also include the ability to reposition the implant in the event that it is mis-deployed. Likewise, there is a need for the capability of holding the device and repositioning the device, using traditional graspers or other ancillary devices to retrieve stones during urological procedures, in conjunction with imaging using an endoscope or cystoscope. The present disclosure addresses these and other needs.

### SUMMARY

This disclosure is directed to a controlled release mechanism for a prostatic implant delivery and deployment system. The controlled release mechanism may have a catheter, a prostatic implant disposed within a distal portion of the catheter, a pusher member coaxially disposed within the catheter proximal to the prostatic implant, a disengageable connection between the pusher member and the prostatic implant and a control member extending between the disengageable connection and a proximal end of the catheter.

In one aspect, the pusher member may be an elongated tubular member.

In one aspect, the pusher member may be a pusher wire and a pusher head such that the disengageable connection is between the prostatic implant and the pusher head.

In one aspect, the disengageable connection may be a wire latch removably disposed in a lumen of the prostatic implant and a suture looping from the pusher member around the wire latch. The pusher member may be a pusher wire and a pusher head. The suture may be secured to the pusher head. The pusher head may have a distal portion and a proximal portion such that the suture is secured to the proximal portion of the pusher head. The suture may be a continuous loop encircling the pusher wire. The pusher head may be conical with a tapered region formed by a plurality of supports.

In one aspect, the disengageable connection may be a fastener at a proximal end of the prostatic implant and a tilt locker secured to the control member such that the control member is a pre-shaped wire.

In one aspect, the disengageable connection may be a fastener at a proximal end of the prostatic implant and a chamfer locker secured to the control member.

In one aspect, the disengageable connection may be a fastener at a proximal end of the prostatic implant and a tube anchor secured to the control member.

In one aspect, the disengageable connection may be hooks extending from the pusher member such that the hooks are maintained in an outward configuration when supported by the control member.

In one aspect, the disengageable connection may be a lock wire extending into an inner diameter of the prostatic implant having an aperture at a distal end and the control member is a loop routed around a structure of the prostatic implant and through the aperture.

In one aspect, the disengageable connection may be a suture connected to the control member and looped through an aperture formed in a proximal portion of the prostatic implant.

This disclosure also includes a method for deploying and disengaging a prostatic implant. The method may involve providing a catheter having a prostatic implant disposed within a distal portion of the catheter, a pusher member coaxially disposed within the catheter proximal to the prostatic implant and a disengageable connection between the pusher member and the prostatic implant. The pusher member may be advanced distally until the prostatic implant is beyond a distal end of the catheter. A control member coupled to the disengageable connection may be actuated at a proximal end of the catheter to release the prostatic implant

In one aspect, actuating the control member may involve withdrawing a wire latch from a lumen of the prostatic implant to disengage a suture looping from the pusher member around the wire latch.

In one aspect, actuating the control member may involve disengaging a lock member associated with the pusher member from a fastener at a proximal end of the prostatic implant.

In one aspect, hooks extending from the pusher member are maintained in an outward configuration by the control member in a supporting position to engage corresponding notches in a proximal portion of the prostatic implant such that actuating the control member may involve withdrawing the control member from the supporting position.

In one aspect, actuating the control member may involve compressing and withdrawing an expandable lock through a proximal neck of the prostatic implant.

In one aspect, a suture loops from an aperture at a distal end of a lock wire by extending into an inner diameter of the prostatic implant and around a structure of the prostatic implant such that actuating the control member may involve withdrawing the suture.

In one aspect, actuating the control member may involve withdrawing the control member to disengage a suture looped through an aperture formed in a proximal portion of the prostatic implant. Withdrawing the control member may break the suture.

This disclosure also includes a handle for a prostatic implant delivery and deployment system. The handle may have a handle body, a plunger configured to be coupled to a pusher member of a controlled release mechanism and an actuator configured to be coupled to a control member of the controlled release mechanism. Manipulation of the plunger is configured to cause the pusher member to deploy a prostatic implant from the controlled release mechanism. The actuator is configured to withdraw the control member to disengage the prostatic implant from the controlled release mechanism when the prostatic implant is beyond a distal end of the controlled release mechanism.

In one aspect, a lock may prevent operation of the actuator until the plunger has been fully advanced during deployment of the prostatic implant.

In one aspect, the plunger may be configured to provide tactile feedback during different stages of prostatic implant deployment.

In one aspect, advancement of the plunger may be configured to be irreversible after advancement of the plunger beyond a predetermined position.

In one aspect, the actuator may be configured to operate automatically when the plunger has been fully advanced.

In one aspect, the handle may also have a release set linkage to accelerate at least one of plunger advancement and actuator operation. The release set may be configured to provide a bail-out function.

In one aspect, the handle also includes a telescoping cylinder controlled by a rotating knob rib that is configured to vary an exposed length of a catheter by extending and retracting. The telescoping cylinder and the rotating knob rib may be coupled by projections on an outer surface of the telescoping cylinder that engage a screw thread formed on an inner surface of the knob rib.

This disclosure also includes a method for deploying and disengaging a prostatic implant. The method may involve providing a controlled release mechanism and a handle, advancing a plunger of the handle that is coupled to a pusher member of a controlled release mechanism to deploy the prostatic implant from the controlled release mechanism and operating an actuator of the handle that is coupled to a control member of the controlled release mechanism to withdraw the control member and disengage the prostatic implant from the controlled release mechanism when the prostatic implant is beyond a distal end of the controlled release mechanism.

In one aspect, operation of the actuator may occur automatically when the plunger has been fully advanced.

In one aspect, tactile feedback regarding stages of deployment of the prostatic implant may be provided during advancement of the plunger.

In one aspect, an exposed length of a catheter may be adjusted by extending and retracting a telescoping cylinder of the handle. The exposed length of the catheter may be adjusted to match dimensions of a working channel of a cystoscope.

This disclosure also includes an irrigation system for a prostatic implant delivery and deployment system. The irrigation system may have a handle, a catheter coupled to the handle by a catheter hub comprising at least one inlet hole, a catheter hub seal, a cylinder secured to the handle that encompasses a proximal portion of the catheter and a liquid inlet in fluid communication with an interior of the cylinder for receiving irrigation fluid.

In one aspect, the cylinder may be telescoping with respect to the handle to vary a length of the catheter exposed from the cylinder.

In one aspect, the irrigation system may have a first irrigation path defined by flow of irrigation fluid into the liquid inlet, through the interior of the cylinder, into the at least one inlet hole of the catheter hub and through an inner channel of the catheter and, when the catheter is disposed within a working channel of a cystoscope, a second irrigation path defined by flow of irrigation fluid into the liquid inlet, through the interior of the cylinder and into an outer lumen formed by an outer diameter of the catheter and the inner diameter of the cystoscope working channel.

This disclosure further includes a method for employing a prostatic implant delivery and deployment system. The method may involve providing a handle having a catheter coupled to the handle by a catheter hub comprising at least one inlet hole, a catheter hub seal, a cylinder secured to the handle that encompasses a proximal portion of the catheter and a liquid inlet in fluid communication with an interior of the cylinder and supplying irrigation fluid through the liquid inlet.

In one aspect, the catheter may be advanced through a working channel of a cystoscope to provide a first irrigation path defined by flow of irrigation fluid into the liquid inlet, through the interior of the cylinder, into the at least one inlet hole of the catheter hub and through an inner channel of the catheter and a second irrigation path defined by flow of irrigation fluid into the liquid inlet, through the interior of the cylinder and into an outer lumen formed by an outer diameter of the catheter and an inner diameter of the cystoscope working channel.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 is a cross-section of the male anatomy comprising the lower portion of the bladder, and the prostatic urethra in a physiological configuration typical of a patient suffering from BPH, showing placement of an implant that may be disposed using the devices and systems of this disclosure in the prostatic urethra and engaging prostatic tissue on either side thereof between the bladder neck opening and the verumontanum according to an embodiment.

FIG. 2 schematically depicts a system for deploying and disengaging a prostatic implant according to an embodiment.

FIG. 3 schematically depicts use of the system with a cystoscope according to an embodiment.

FIGS. 4-6 schematically depict a controlled release mechanism employing a pusher member according to an embodiment.

FIG. 7 schematically depicts a controlled release mechanism employing a pusher head and pusher wire according to an embodiment.

FIGS. 8-10 schematically depict a controlled release mechanism employing a pusher head with a wire latch lumen according to an embodiment.

FIGS. 11-12 schematically depict a controlled release mechanism employing a two part pusher head according to an embodiment.

FIGS. 13-14 schematically depict a controlled release mechanism employing a knotted suture loop according to an embodiment.

FIGS. 15-16 schematically depict a controlled release mechanism employing a continuous suture loop according to an embodiment.

FIGS. 17-20 schematically depict a controlled release mechanism employing a conical pusher head according to an embodiment.

FIGS. 21-22 schematically depict a controlled release mechanism employing a tilting locker according to an embodiment.

FIGS. 23-24 schematically depict a controlled release mechanism employing a chamfered locker according to an embodiment.

FIGS. 25-26 schematically depict a controlled release mechanism employing a tube anchor according to an embodiment.

FIGS. 27-28 schematically depict a controlled release mechanism employing a pusher member with hook extensions according to an embodiment.

FIGS. 29-30 schematically depict a controlled release mechanism employing an expandable locker member according to an embodiment.

FIGS. 31-32 schematically depict alternative embodiments of an expandable locker.

FIG. 33 schematically depicts a controlled release mechanism employing a lock wire according to an embodiment.

FIGS. 34-35 schematically depict a controlled release mechanism employing a suture lock according to an embodiment.

FIGS. 36-38 schematically depict a handle mechanism for deploying and disengaging a prostatic implant according to an embodiment.

FIGS. 39-41 schematically depict different stages of deployment according to an embodiment.

FIGS. 42-43 schematically depict disengagement of a prostatic implant using a handle mechanism according to an embodiment.

FIG. 44 schematically depicts a release set for a handle mechanism according to an embodiment.

FIGS. 45-50 schematically depict different stages of deployment using the release set of FIG. 44 according to an embodiment.

FIGS. 51-54 schematically depict a bail-out procedure using the release set of FIG. 44 according to an embodiment.

FIG. 55 schematically depicts a release set for a handle mechanism according to another embodiment.

FIGS. 56-59 schematically depict different stages of deployment using the release set of FIG. 55 according to an embodiment.

FIGS. 60-61 schematically depict a bail-out procedure using the release set of FIG. 55 according to an embodiment.

FIG. 62 schematically depicts a handle mechanism having a telescoping function according to another embodiment.

FIGS. 63-64 schematically depict extension and retraction of a telescoping handle mechanism according to an embodiment.

FIG. 65 schematically depicts a handle mechanism having a dual path irrigation system according to an embodiment.

FIGS. 66-69 schematically depict the two paths of irrigation flow with the handle mechanism of FIG. 65 according to an embodiment.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. Moreover, as used in this specification and the appended claims, the singular forms “a,” “an” and “the” include plural referents unless the content clearly dictates otherwise.

Definitions: The terms “therapeutically effective displacement” or “therapeutically effective retraction” or “therapeutically effective expansion”, are used interchangeably herein and refer to an amount of displacement of prostatic tissue proximate to a restricted area of a urethra sufficient to increase the urethral lumen and treat, ameliorate, or prevent the symptoms of benign prostatic hyperplasia (BPH) or comorbid diseases or conditions, including lower urinary tract symptoms (LUTS), bladder outlet obstruction (BOO), benign prostatic obstruction (BPO), wherein the displacement of prostatic tissues exhibits a detectable therapeutic, prophylactic, or inhibitory effect. The effect can be detected by, for example, an improvement in clinical condition, or reduction in symptoms or absence of co-morbidities. Examples of clinical measures include a decrease in the international prostate symptom score (IPSS), reduction in post-void residual (PVR) volume of urine in the bladder after relief or increase in the maximum urinary flow rate (Qmax) or improvement in quality of life (QoL), improvement in sexual health (sexual health inventory for men or SHIM score, men's sexual health questionnaire or MSHQ score) after treatment. The precise distance or volume of the displacement of prostatic tissue will depend upon the subject's body weight, size, and health; the nature and extent of the enlarged or diseased prostatic condition and the size of the implant selected for placement in the patient.

As used herein, a patient “in need of treatment for BPH” is a patient who would benefit from a reduction in the presence of or resulting symptoms of enlarged prostatic tissue caused by a non-malignant enlarging of the prostate gland and related disorders, including LUTS, urinary outflow obstruction symptoms and luminal narrowing of the prostatic urethra. As used herein, the terms “implant” or “expander” or “device” refer to the prosthetic device that is implanted within the prostatic urethra to relieve LUTS associated or caused by BPH.

As used herein, the terms “tissue engaging” with regard to arms, struts or other extensions of the structure of the implant refers to a length of the physical structure of the implant that engages prostatic tissue along the main portion of the lobes of the organ compressing on the urethra and restraints the tissue from further impingement on the patency of the urethra. “Tissue retracting” refers to the ability of the structure of the implant to exert the requisite force to displace tissue away from the compressed or narrowed urethra. The requisite force could be supplied by the inherent structure of the implant or by the expansion of the implant from the compressed to the expanded configuration, particularly where the implant is fabricated from a shape-memory or super-elastic material having a predetermined expanded configuration designed to engage the hyperplasic prostate tissue and exert the requisite tissue retraction force. The length of a tissue-engaging or tissue-retracting structural feature in contact within these definitions is spaced away from the intra-lobular grooves that run along the length of the prostate surrounding the urethra and requires contact with a length of tissue along the length of the two lateral or lateral and medial lobes.

With respect to orientation of the various structures and anatomical references described herein, the term “proximal” and “distal” are relative to the perspective of the medical professional, such as a urologist, who is manipulating the delivery system of the disclosure to deploy the implants described herein. Accordingly, those features of the delivery system held by the hand of the urologist are at the “proximal” end and the assembled system and the implant, initially in its compressed configuration, is located at the “distal” end of the delivery system.

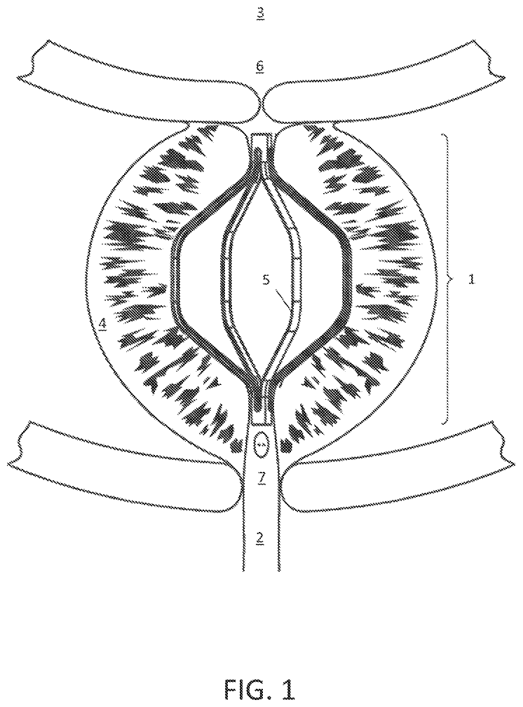

Referring to FIG. 1, a cross-section of the male anatomy shows the prostate gland 1 surrounding the urethra 2. The urethra 2, under normal conditions, provides fluid communication from urine stored in the bladder 3 to be expelled from the body under voluntary muscular control of the external urethral sphincter. Normal or “true” prostate tissue 4 surrounds the urethra 2 and, in the absence of disease, does not impinge on the patency of the urethra 2. In patients suffering from benign prostatic hyperplasia (BPH), the urethra 2 is narrowed by hyperplasic tissue, i.e., prostate tissue 4 that exhibits excess growth towards the urethra 2. This excess of non-cancerous cellular growth leads to the symptoms of BPH described above, including, lower urinary tract symptoms (LUTS) and urinary outflow obstruction, and urinary incontinence. In FIG. 1, an implant 5 delivered using the devices and systems of this disclosure is shown engaging prostate tissue 4 along a length of the implant 5 to restore the patency of the urethra 2 and to permit unimpeded urine flow from the bladder 3. The selective placement of the implant 5 at a target site, between bladder neck opening 6 and verumontanum 7, as shown is an important characteristic so that implant 5 does not puncture, perforate or incise the surrounding tissue. The implant 5 is designed to remain in place within the prostatic urethra 2. The implant 5 does not extend into the urinary bladder 3, where the structural material of the implant 5 could become encrusted or otherwise degraded from constant exposure to urine causing complications and making retrieval more difficult, and the implant 5 does not interfere with the voluntary control of the external urethral sphincter or interfere with sexual functions.

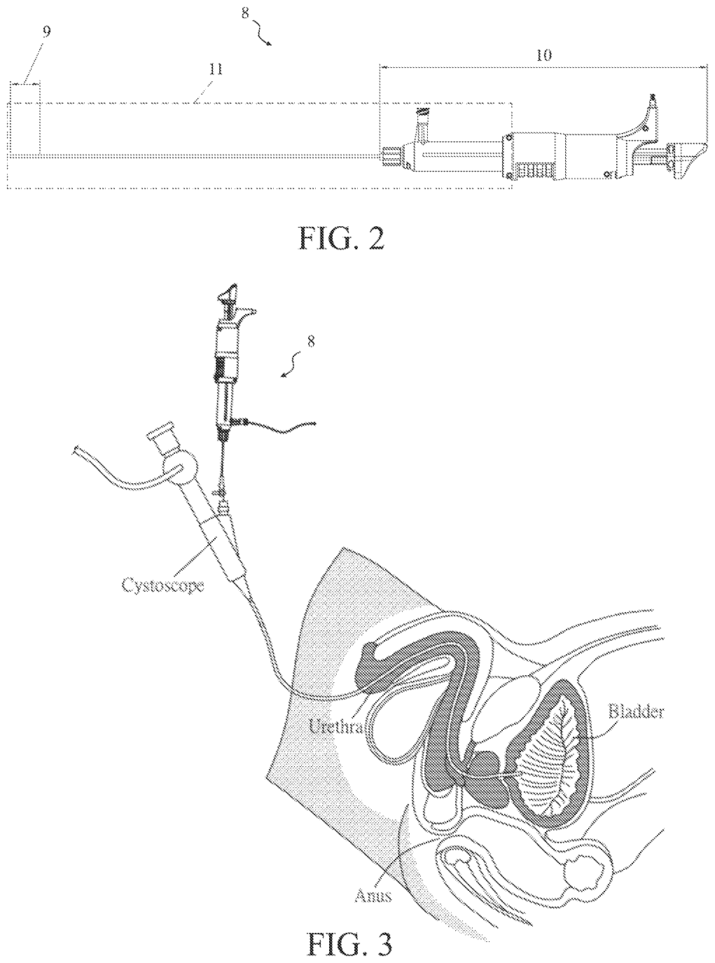

The present disclosure involves a system 8 for delivering and deploying an implant at a desired location in a lumen of a body for the treatment of benign prostatic hyperplasia (BPH) and related lower urinary tract symptoms (LUTS), as exemplified by the embodiment shown in FIG. 2. The system involves a controlled release mechanism 9, a handle mechanism 10 and an irrigation system 11. As an illustration and without limitation, FIG. 3 schematically depicts usage of system 8 for delivering and deploying prostatic implant 5 to relieve benign prostatic hyperplasia (BPH) in conjunction with either a flexible or rigid cystoscope to aid positioning and deploying prostatic implant 5, with the associated irrigation system 11 into suitable location in the prostatic urethra 2. After the positioning, prostatic implant 5 may be released and expanded to restore patency to the prostate gland.

A feature common to embodiments of this disclosure relates to the mechanical coupling of an expandable implant, such as implant 5, to a pusher member that is in turn then mechanically coupled to a plunger in handle mechanism 10. Thus, when the plunger is moved by the user, the pusher member and the expandable implant will be moved accordingly. As will be discussed below, the pusher member may push against and locate the expandable implant to a suitable position in the prostatic urethra. After positioning, a wire latch may be withdrawn such that the expandable implant will be decoupled from the pusher member. As one example, the pusher member may include a pusher head and a pusher wire. The pusher head is mechanically coupled to the plunger through the pusher wire, which could be made of Nitinol, to maximize irrigation flow. Moreover, in another aspect of the present disclosure, (1) the moving or positioning of the pusher member with the expandable implant and (2) the decoupling between the pusher member with the expandable implant may be controlled by a single actuator stroke integrated within handle mechanism 10.

In another aspect of the present disclosure, handle mechanism 10 may feature an outer telescope cylinder coupled with a handle body such that a catheter is fixed to the handle body and passes thought the telescope cylinder. Specifically, the telescope cylinder is extendable and retractable with respect to the handle body such that the length of the catheter exposed out of the telescope cylinder is adjustable. With such adjustable length of the catheter, the present invention is conveniently compatible with different types of commercially-available cystoscopes which may require different dimensions.

Furthermore, in another aspect of the present disclosure, an irrigation system is provided with at least one fluid path for facilitating visualization under cystoscopy during the operation. A suitable irrigation fluid, such as saline, may be directed either through an inner channel of the catheter or through an outer lumen of the catheter.

Although the present disclosure has been illustrated and described with reference to the following embodiments, it is to be understood that the disclosure is not to be limited to the disclosed embodiments, but on the contrary, is intended to cover various modifications and equivalent arrangements included within the spirit and scope of the present disclosure.

### Controlled Release Mechanism

The disclosed release mechanism involves a configuration between a prostatic implant and engaging elements. For example, the engaging element may be a releasable tether of suture material coupled to a proximal end of the prostatic implant. The engaging elements may also include interlocking features at a distal end of the pusher member and a proximal end of the prostatic implant. Still further, the engaging element may be a wire or suture routed through apertures formed in tissue-engaging portions of the prostatic implant that constrains the prostatic implant when tensioned or may be a wire routed around tissue-engaging portions of the prostatic implant that constrains the prostatic implant when tensioned. This section of the disclosure relates to several embodiments regarding the controlled release mechanism.

1. Wire Latch

Embodiments of the disclosure includes a controlled release mechanism employing a wire latch to hold, push, lock and unlock a prostatic implant as shown in FIG. 4. The controlled release mechanism comprises prostatic implant 5, a catheter 12 connecting with handle mechanism of FIG. 2 for example, a pusher member 13, which could be a tube, connecting with a plunger (not shown) in handle mechanism 10, a suture 14 connecting with pusher member 13 or handle mechanism 10, and a control member such as a wire latch 15 connecting with an actuator on the plunger and routed through a lumen in pusher member 13. A recess 16 is formed in the sidewall of the proximal end of prostatic implant 5 and connects to an internal lumen. As shown, prostatic implant 5 and pusher member 13 are engaged by wire latch 15, which passes through the lumen of the prostatic implant 11, and a loop of suture 14 the extends from plunger shaft 13 into recess 16.

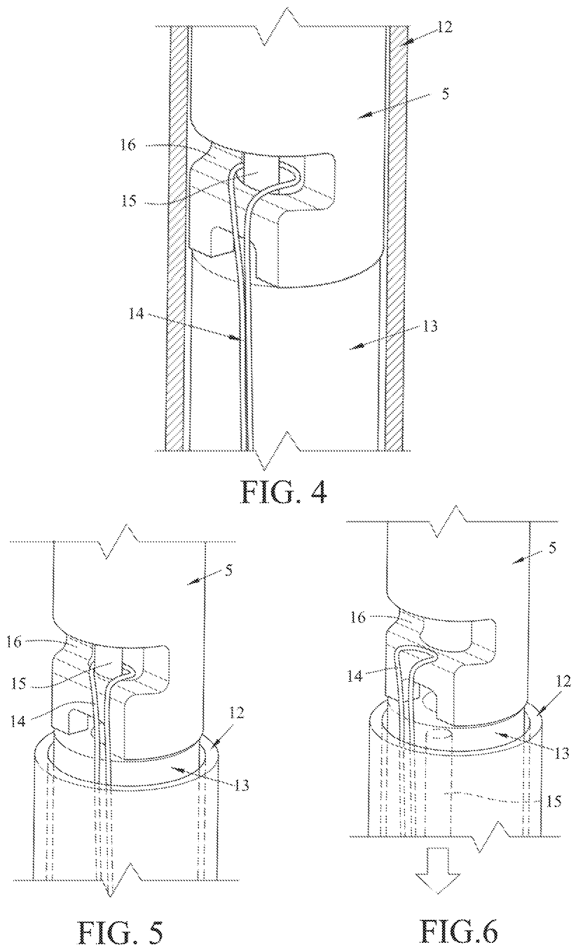

An exemplary technique for employing this embodiment is schematically depicted in FIGS. 5 and 6. Prior to deployment, prostatic implant 5 is disposed within a distal end of catheter 12 and locks with a loop of suture 14 and wire latch 15 according to the above discussion. Correspondingly, prostatic implant 5 may be released by a deployment step including pushing prostatic implant 5 out the distal end of catheter 12 as represented by FIG. 5. At this stage, wire latch 15 still engages suture 14 loop with prostatic implant 5. Next, a prostatic implant disengaging step may be formed as indicated by FIG. 6. Notably, retracting wire latch 15 with the actuator of the plunger of handle mechanism 10 releases the loop of suture 14 to disengage prostatic implant 5.

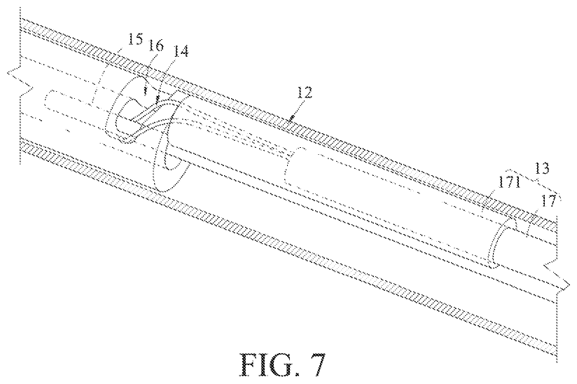

Implementations of this controlled release mechanism may include usage of a pusher wire as indicated in the embodiment of FIG. 7 showing prostatic implant 5, catheter 12 connecting with handle mechanism of FIG. 2 for example, pusher member 13 which here includes a pusher wire 17 and a pusher head 171, suture 14 in a loop profile connecting to the pusher head 1711 at the distal end of pusher member 13, and wire latch 15, which is connected with an actuator in the handle mechanism. Pusher wire 17 extends through pusher head 171 and connects with a plunger in the handle mechanism. Pusher member 13 and wire latch 15 may adjacent and are individually operable. Prostatic implant 5 is retained by pusher head 171 through the engagement of wire latch 15, which as noted above is retractable from the lumen of prostatic implant 5, and the loop of suture 14 that extends within recess 16. In one embodiment, when the plunger in the handle mechanism is moved, pusher wire 17 will latterly transmit the force to push or pull pusher head 171. Because pusher head 171 and prostatic implant 5 are engaged at this stage, prostatic implant 5 may be easily positioned or deployed by manipulation of pusher member 13.

In comparison with the embodiment of FIGS. 4-6 in which pusher member 13 could be implemented with a tube, pusher wire 17 is instead used to couple to pusher head 171, which may have an outer diameter greater than the outer diameter of pusher wire 17. Pusher head 171 may also be formed as part of pusher wire 17. To improve irrigation functionality and/or to facilitate clinical operation, pusher wire 17 may be a flexible metal such as Nitinol. Since, the cross-sectional area of pusher wire 17 exhibits a lower profile than a tube, such embodiments have greater space between catheter 12 and pusher member 13 which may aid cystoscope visualization.

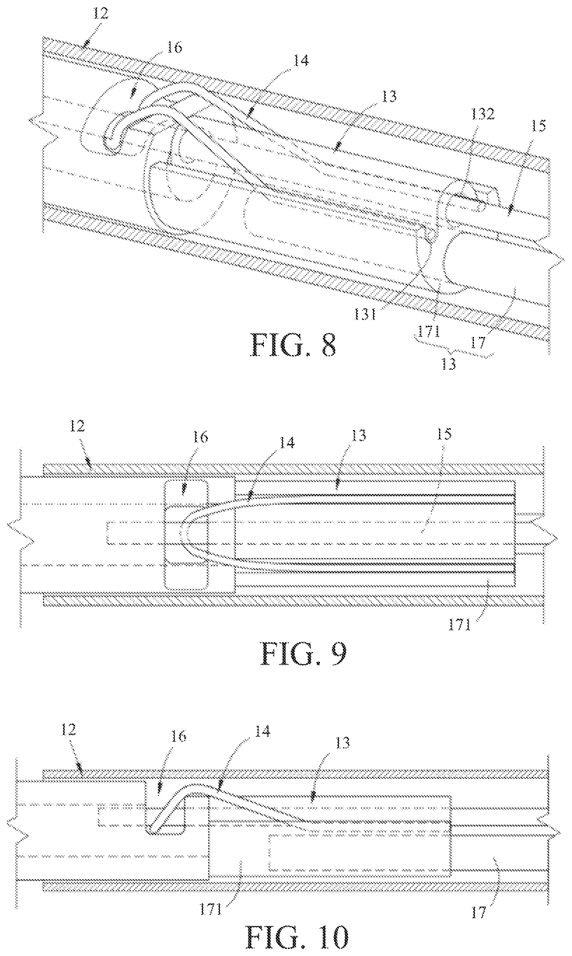

Another embodiment of controlled release mechanism 9 is shown in FIGS. 8-10, with elevational, top and side views, respectively. In these views, controlled release mechanism 9 is shown with prostatic implant 5, catheter 12 (connecting with handle mechanism of FIG. 2 for example), pusher head 171 of pusher wire 17 implementing pusher member 13, loop of suture 14 connecting to pusher head 171 and wire latch 15 (connecting with an actuator the handle mechanism 10). Pusher head 171 has grooves 131 and a pusher head lumen 132 which aligns with the lumen of prostatic implant 5. Suture 14 is disposed in grooves 131 in loop profile. Wire latch 15 extends through pusher head lumen 132, the lumen of prostatic implant 5 and the loop of suture 14 to engage prostatic implant 5 and retain it with pusher head 171.

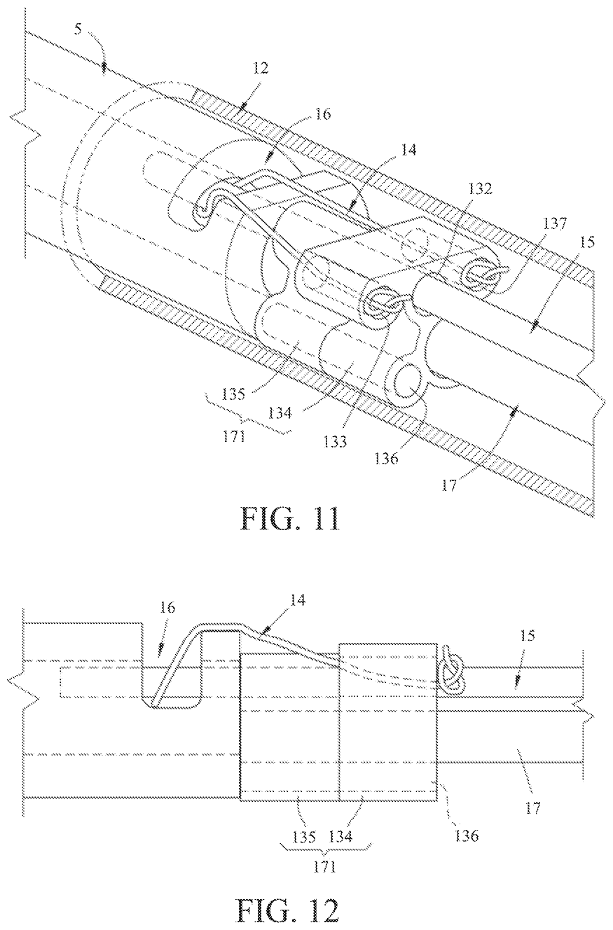

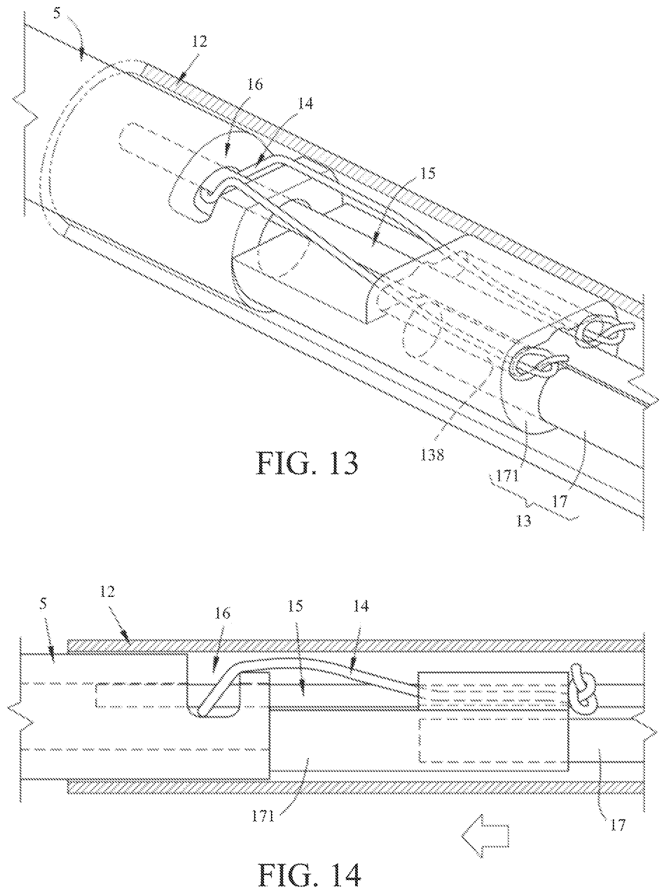

Yet another embodiment of controlled release mechanism 9 is shown in FIGS. 11-12, with elevational and side views respectively. In these views, controlled release mechanism 9 is shown with prostatic implant 5, catheter 12 (connecting with handle mechanism of FIG. 2 for example), pusher head 171 of pusher wire 17 implementing pusher member 13, loop of suture 14 connecting to pusher head 171 and wire latch 15 (connecting with an actuator the handle mechanism 10). Pusher head 171 has a first pusher head 134 and a second pusher head 135. Second pusher head 135 is located at a distal side of first pusher head 134 is connected to first pusher head 134 with anti-rotation pins 136 to maintain their rotational alignment. First pusher head 134 has two suture anchor holes 137 so that respective ends suture 14 may be passed through and fixed at each proximal opening by a suture knot 133 to form the loop that is engaged by wire latch 15. Pusher wire 17 of pusher member 13 connects with the proximal end of first pusher head 171. In one example, suture 14 may be a Nitinol wire and fixed on the first pusher head 134. Each of first and second pusher heads 134, 135 has pusher head lumen 132 that align with the lumen of prostatic implant 5, so that wire latch 15 can pass through pusher head lumens 132, the lumen of prostatic implant 5 and the loop of suture 14 to engage prostatic implant 5 with pusher head 171 within recess 16. As with the other embodiments of this disclosure, prostatic implant 5 may be controlled by the pusher head 171 or pusher wire 17 during deployment due to the engagement.

A further embodiment of controlled release mechanism 9 is shown in FIGS. 13-14, with elevational and side views respectively. In these views, controlled release mechanism 9 is shown with prostatic implant 5, catheter 12 (connecting with handle mechanism of FIG. 2 for example), pusher head 171 of pusher wire 17 implementing pusher member 13, loop of suture 14 connecting to pusher head 171 and wire latch 15 (connecting with an actuator the handle mechanism 10). Here, pusher head 171 of pusher member 3 has a suture anchor slot portion 138 at its proximal end so that suture 14 may be connected with pusher head 171 by passing the two ends of suture 14 through suture anchor slot portion 138 and tying knots, so that suture 14 is formed with a loop profile for engagement by wire latch 15. Correspondingly, wire latch 15 can also pass through the suture anchor slot part 138 into the lumen of prostatic implant 5 in a manner similar to the other embodiments. Pusher wire 17 of pusher member 3 connects with the proximal end of pusher head 171. Prostatic implant 5 and pusher head 171 are engaged as noted above, with wire latch 15 extending through the lumen of prostatic implant 5 and the loop of suture 14 within recess 16, so that prostatic implant 5 may be controlled by the movement of pusher head 171 or pusher wire 17.

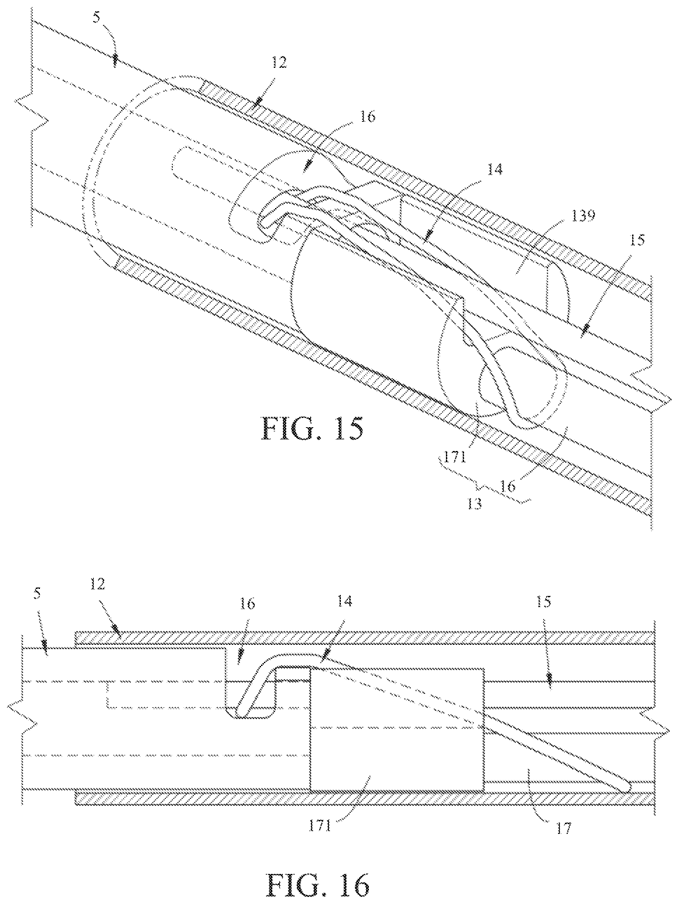

Yet another embodiment of controlled release mechanism 9 is shown in FIGS. 15-16, with elevational and side views respectively. In these views, controlled release mechanism 9 is shown with prostatic implant 5, catheter 12 (connecting with handle mechanism of FIG. 2 for example), pusher head 171 of pusher wire 17 implementing pusher member 13, loop of suture 14 connecting to pusher head 171 and wire latch 15 (connecting with an actuator the handle mechanism 10). In this embodiment, pusher head 171 of pusher member 13 has a groove 139 for accommodating suture 14 and wire latch 15. Suture 14 may be a continuous loop or ring that encircles wire latch 15 and pusher wire 17. Pusher wire 17 connects with the proximal end of pusher head 171. Prostatic implant 5 and pusher head 171 are engaged as noted above, with wire latch 15 extending through the lumen of prostatic implant 5 and the loop of suture 14 within recess 16, so that prostatic implant 5 may be controlled by the movement of pusher head 171 or pusher wire 17.

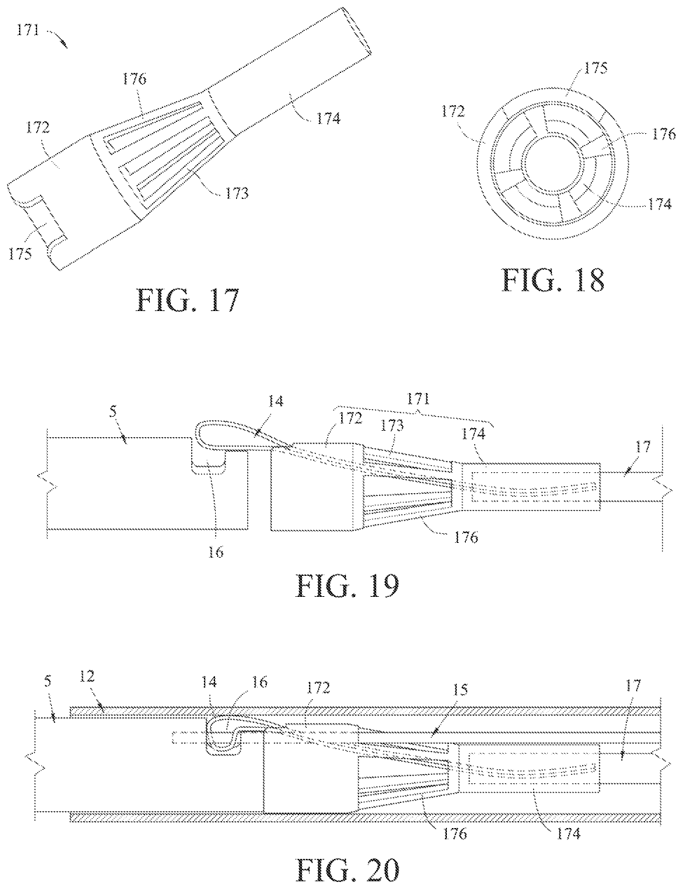

A still further embodiment of controlled release mechanism 9 is shown in FIGS. 17-20. In these views, controlled release mechanism 9 is shown with prostatic implant 5, catheter 12 (connecting with handle mechanism of FIG. 2 for example), pusher head 171 of pusher wire 17 implementing pusher member 13, loop of suture 14 connecting to pusher head 171 and wire latch 15 (connecting with an actuator the handle mechanism 10). A detail elevational and a proximal end view of pusher head 17 are depicted in FIGS. 17 and 18, respectively. As shown, pusher head 171 is conical and with a distal end portion 172, a flared region 173 and a proximal neck 174. A gap 175 on distal end portion 172 accommodates suture 14, which is configured as a loop where it extends from pusher head 171 as shown in FIG. 19. Flared region 173 is formed by support bars 176 that are sized to allow wire latch 15 to pass between. Neck region 174 connects suture 14 and pusher wire 17, so that prostatic implant 5 may be controlled by pusher head 171 or the pusher member 3 when engaged as shown in FIG. 20 in a similar manner to that described above.

2. Prostatic Implant Fastener

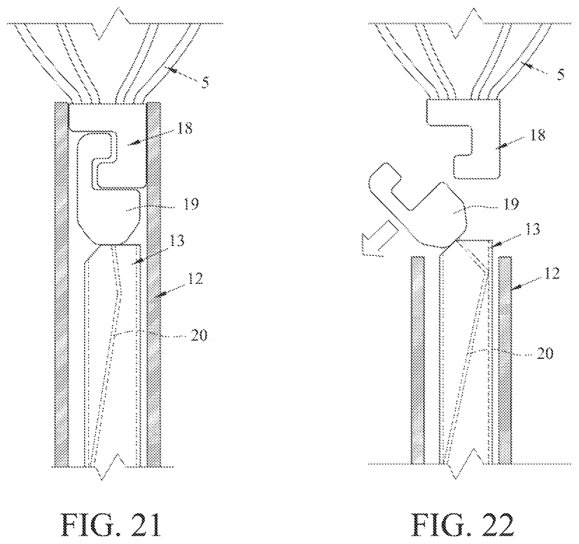

Embodiments of the disclosure include a controlled release mechanism employing a lock member that engages a fastening structure to hold, push, lock and unlock a prostatic implant as shown in FIGS. 21 and 22. The controlled release mechanism includes a fastener 18 at the proximal end of prostatic implant 5, catheter 12 connecting with handle mechanism of FIG. 2 for example, pusher member 13 connecting with a plunger (not shown) in handle mechanism 10 and a tilt locker 19 connected to a control member such as a pre-shaped wire 20 that extends through pusher member 13 so that it can be actuated at handle mechanism 10. Tilt locker 19 and fastener 18 are configured to engage when axially aligned, such as when tilt locker 19 is restrained within catheter 12 as shown in FIG. 21. Upon full deployment of prostatic implant 5 as shown in FIG. 22, the end of pre-shaped wire 20 connected to tilt locker 19 deflects automatically when unconstrained by catheter 12 to disengage from fastener 18 of prostatic implant 5.

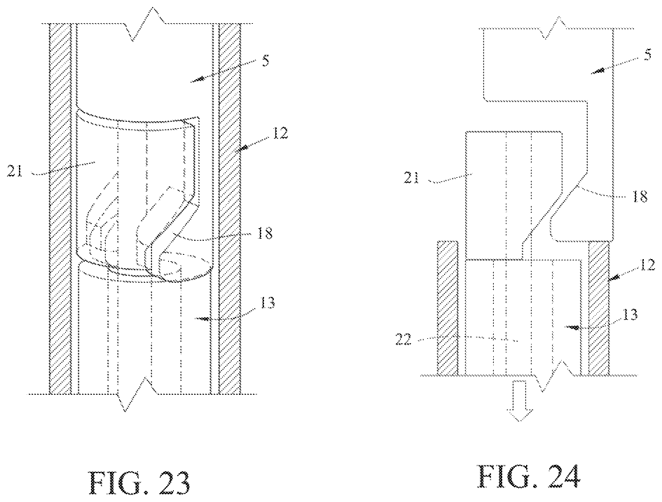

Another embodiment includes a controlled release mechanism employing a chamfer lock member as shown in FIGS. 23 and 24. The controlled release mechanism includes fastener 18 at the proximal end of prostatic implant 5, catheter 12 connecting with handle mechanism of FIG. 2 for example, pusher member 13 connecting with a plunger (not shown) in handle mechanism 10 and a chamfer locker 21 that is either formed at the distal end of pusher member 13 or is connected to a control member such as control wire 22 that extends through pusher member 13 so that it can be actuated at handle mechanism 10. Chamfer locker 21 engages with fastener 18 when axially aligned due to contact surfaces having opposing chamfers or wedge configurations so prostatic implant 5 is retained when chamfer locker 21 is restrained within catheter 12 as shown in FIG. 23. Upon full deployment of prostatic implant 5, the chamfered surfaces allow chamfer locker 21 to slide and release fastener 18 of prostatic implant 5 as shown in FIG. 24.

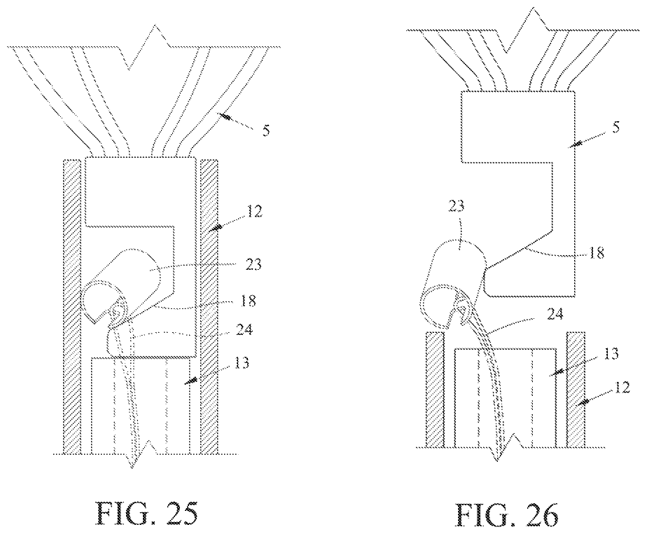

A further embodiment includes a controlled release mechanism employing an anchor lock member as shown in FIGS. 25 and 26. The controlled release mechanism includes fastener 18 at the proximal end of prostatic implant 5, catheter 12 connecting with handle mechanism 10 of FIG. 2 for example, pusher member 13 connecting with a plunger (not shown) in handle mechanism 10 and an anchor 23, configured as a tube in this embodiment but other configurations may be used, secured to a control member such as a control suture 24. When anchor 23 is engaged with fastener 18 and constrained within catheter 12), prostatic implant 5 is retained and can controlled with control suture 24 and pusher member 13 before being released as anchor 23 will slide against the chamfer of fastener 18 smoothly when unconstrained.

3. Hook

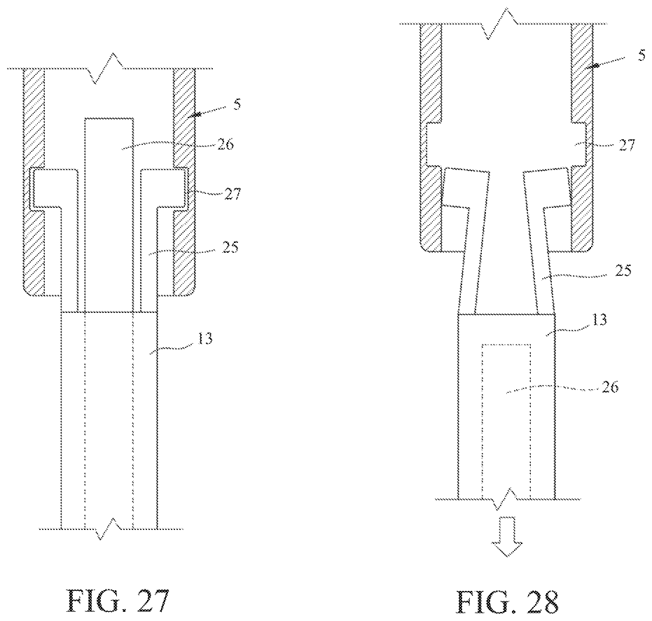

Embodiments of the disclosure include a controlled release mechanism employing hooks to hold, push, lock and unlock a prostatic implant as shown in FIGS. 27 and 28. The controlled release mechanism features catheter 12 connecting with handle mechanism of FIG. 2 for example, pusher member 13 connecting with a plunger (not shown) in handle mechanism 10 and having hooks 25 extending from its distal end that are maintained in outward configuration by a coaxially extending control member 26 by supporting hooks 25 when longitudinally adjacent. Prostatic implant 5 has notches 27 that correspond to and are engageable by hooks 25, although windows, recess or other similar structures may be employed instead. Accordingly, when control member 26 is in a support position coextensive with hooks 25 as shown in FIG. 27, they are maintained in engagement with notches 27. Hooks 25 are designed to flex with respect to pusher member 13, such as being made by Nitinol or other flexible material, and may be pre-shaped to be inward bending to deflect inwards upon withdrawal of control member 26 to disengage from notches 27 and release prostatic implant 5 to complete deployment as per FIG. 28.

4. Expandable Lock

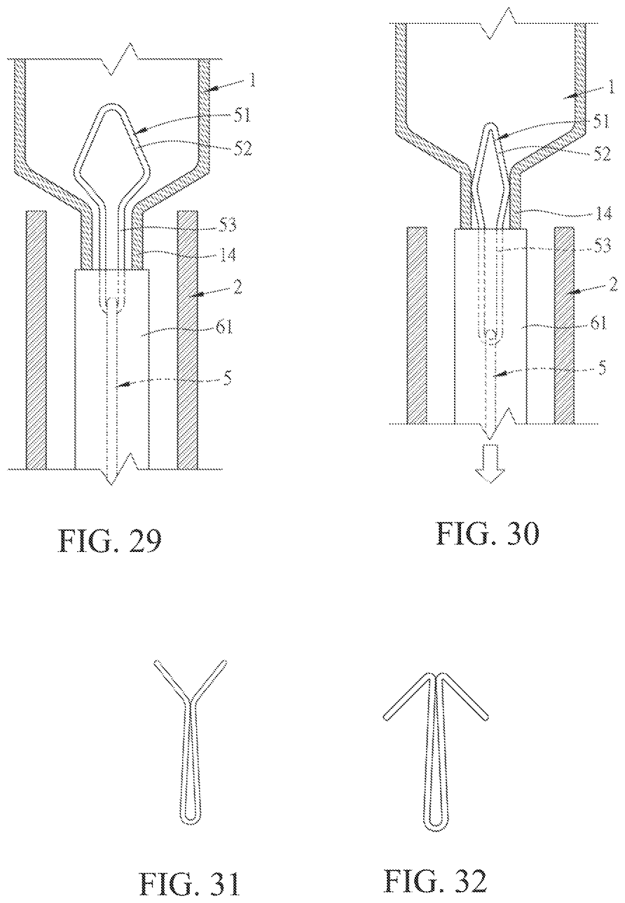

Other embodiments of the disclosure include a controlled release mechanism employing an expandable member to hold, push, lock and unlock a prostatic implant as shown in FIGS. 29 and 30. The controlled release mechanism features catheter 12 connecting with handle mechanism of FIG. 2 for example, pusher member 13 connecting with a plunger (not shown) in handle mechanism 10 and a lock 28 having an expandable head portion 29 and a support portion 30 sized to be able to be withdrawn into pusher member 13. Head portion 29 has a larger diameter when expanded than a neck 31 of prostatic implant 5 and therefore can engage and retain prostatic implant 5 so that it can be controlled with pusher member 13 as indicated by FIG. 29. A control member 32 is secured to support portion 30 and when deployment is to be completed, may be used to compress and withdraw expandable lock 28 at least proximal of neck 31 to release prostatic implant 5. It will be appreciated that that expandable lock 28 can employ different configurations of head portion 29 as long as it has flexible and/or expandable characteristics and can anchor prostatic implant 5, with FIGS. 31 and 32 providing non-limiting examples.

5. Lock Wire

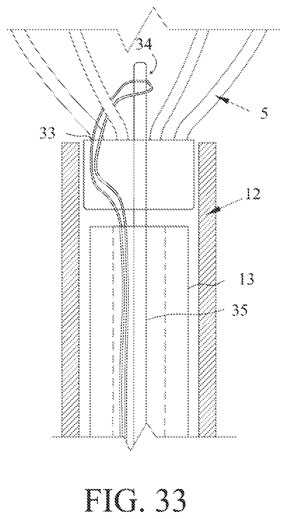

Still other embodiments of the disclosure include a controlled release mechanism employing a lock wire and suture to hold, push, lock and unlock a prostatic implant as shown in FIG. 33. The controlled release mechanism features catheter 12 connecting with handle mechanism of FIG. 2 for example, pusher member 13 connecting with a plunger (not shown) in handle mechanism 10 and a lock wire 33 having an aperture 34 adjacent its distal end. Lock wire 33 extends from the distal end of pusher member 13 into an inner diameter of prostatic implant 5. A control member such as suture 35 may be looped through prostatic implant 5 and aperture 34 and extends through pusher member 13 so that it can be actuated at handle mechanism 10. Suture 35 is routed around a structure of prostatic implant 5, such as a strut or support arm so that tension applied to suture 35 engages and retains prostatic implant 5 so that it can be controlled with pusher member 13. As one non-limiting illustration, suture 35 can be implemented with a 0.005″ Nitinol wire.

6. Suture Lock

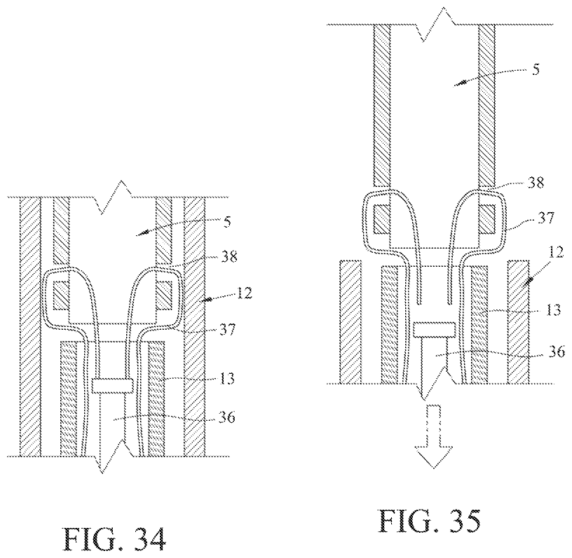

Further embodiments of the disclosure include a controlled release mechanism employing a suture and control to hold, push, lock and unlock a prostatic implant as shown in FIGS. 34 and 35. The controlled release mechanism features catheter 12 connecting with handle mechanism of FIG. 2 for example, pusher member 13 connecting with a plunger (not shown) in handle mechanism 10 and a control member 36 to which ends of suture 37 are secured. Suture 37 loops through at least two apertures 38 formed in a proximal end of prostatic implant 5 to engage and retain prostatic implant 5 so that it can be controlled with pusher member 13 as indicated in FIG. 34. Control member 36 extends through pusher member 13 as shown so that it can be actuated at handle mechanism 10. When deployment of prostatic implant 5 is to be completed after using pusher member 13 to eject prostatic implant 5 from the distal end of catheter 12, actuation of control member 36 may be performed to disengage suture 37 by breaking or withdrawing suture 37 according to FIG. 35.

### Handle Mechanism

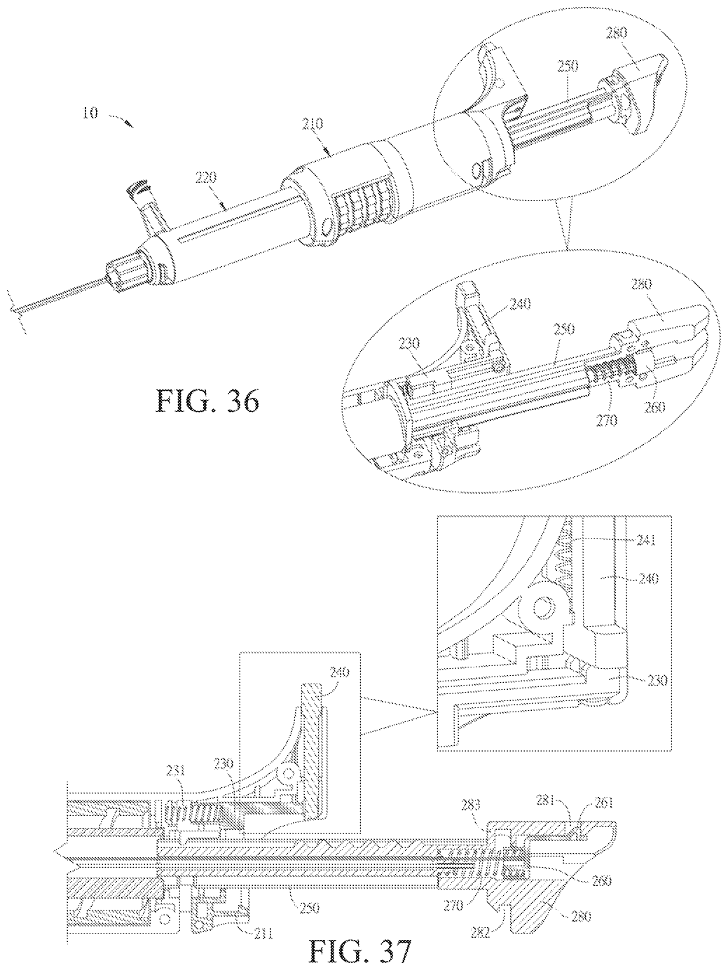

Referring back to FIG. 2, this disclosure also includes handle mechanism 10 that is used in conjunction with the control release mechanisms discussed above for delivering and deploying prostatic implant 5 to relieve benign prostatic hyperplasia (BPH). The following materials discuss handle mechanism 10 and its features of deploying and/or repositioning the expandable implant. In one aspect, the disclosure provides handle mechanism 10 as shown in FIG. 36 to deploy and release prostatic implant 5 and adjust the length of catheter 12 to fit different brands and configurations of cystoscopes when needed. In particular, handle mechanism includes a handle body 210, a telescopic cylinder 220, a button lock 230, a release button 240, a plunger 250 connecting with pusher member 13 (not shown in this view), which in some embodiments is implemented as pusher wire 17 and pusher head 171, an actuator 260 connecting with a control member (not shown in this view), such as wire latch 15, an actuator spring 270 configured to retract the control member and a plunger grip 280. Thus, in relation to the embodiments described above, movement of plunger 250 may be used to cause a corresponding movement of pusher member 13 or pusher head 171 via pusher wire 17. Actuator 260 is moveable on plunger 250 and may be locked through the engagement of a fastener 261 into a top perforation 281 as shown in FIG. 37.

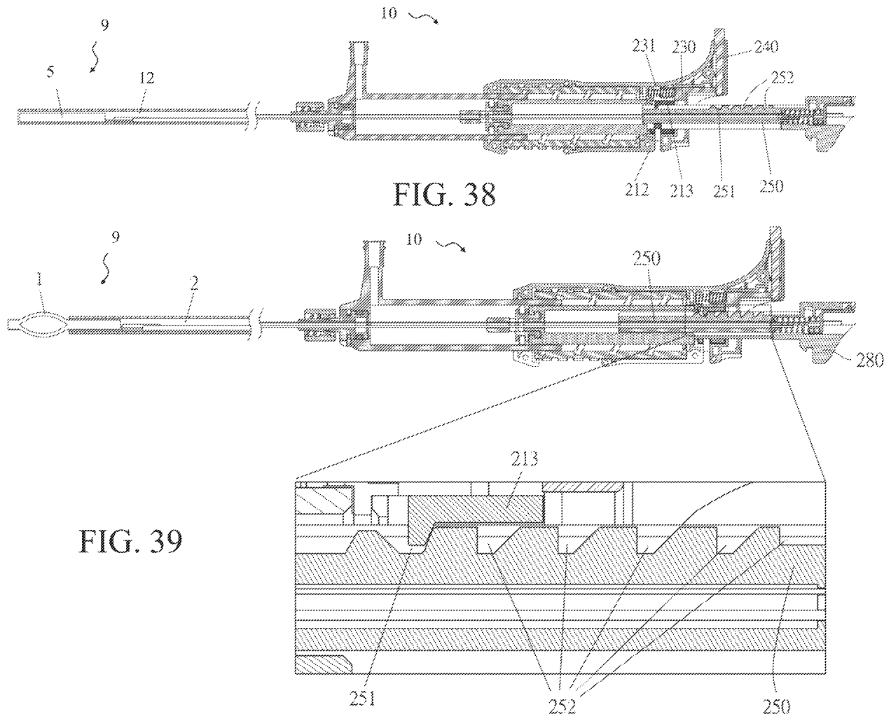

Handle mechanism 10 may be employed to first deploy and then disengage prostatic implement using the control release mechanisms noted above as part of sequential stages. The distal end of pusher member 13 or pusher head 171 is coupled to plunger 250 so that prostatic implant 5 can be pushed out the distal end of catheter 12 and then disengaged from pusher member 13 or pusher head 171. One or more slots 251, 252 on the upper surface of the plunger 250 provide tactile signals for the different stages of deployment. A plunger bushing 213 is configured to engage with the upper surface and slides on the upper surface the plunger 250 as shown in FIG. 38. The interaction between plunger bushing 213 and the upper surface plunger 250 not only provides tactile feedback to indicate different deployment stages, but also provides an irreversible mechanism after certain level of deployment and prevent plunger 250 from pulling back.

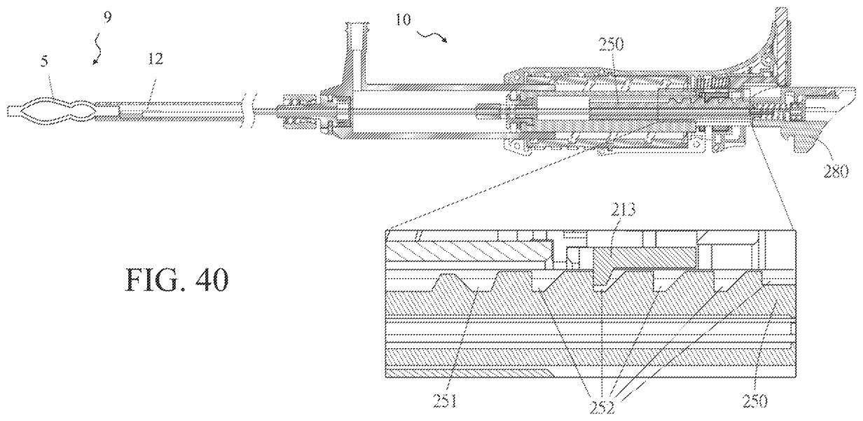

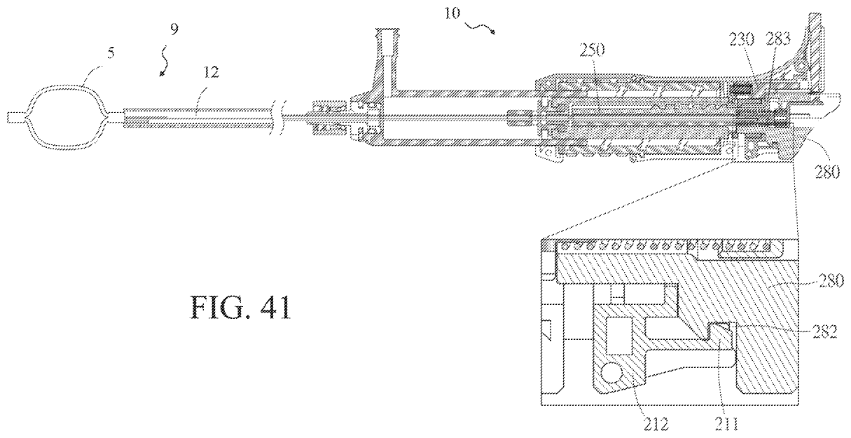

During an initial stage of deployment, release button 240 is locked by the button lock 230 as biased by a lock spring 231 to prevent mis-triggering during deployment of the plunger. Prostatic implant 5 is fully collapsed and disposed within catheter 12 as shown in FIG. 38. Referring next to FIG. 39, a user may hold handle body 210 and push plunger grip 280 after the distal end of catheter 12 is positioned at the target location for deployment of prostatic implant 5. A projection of plunger bushing 213 falls into first slot 251 of plunger 250 providing tactile feedback to the user and indicating one-half stroke has been performed. At this stage, prostatic implant 5 is still partially disposed in catheter 12. As shown in the detail inset, both the distal and proximal surfaces of first slot 251 are chamfered or sloped to allow plunger 250 to move proximally and be drawn back. Since prostatic implant 5 is still engaged with pusher member 13 or pusher head 171, deployment is still reversible at this stage. Deployment from one-half to a full stroke is schematically depicted in FIG. 40. As shown, further manipulation of plunger grip 280 to advance plunger 250 causes plunger bushing 213 to engage second and subsequent slots 252, with each detent providing tactile feedback to the user due to the plastic arm on plunger bushing 213 continuously engaging and disengaging the sequential slots 252. Notably, slots 252 are configured to prevent withdrawal of plunger 250 by having a 90-degree wall on the distal surfaces. Correspondingly, once plunger 250 has been advanced past the one-half stroke position, prostatic implant 5 plunger bushing 213 prevents draw back. Although the motion of plunger 250 is irreversible once the one-half stroke position is achieved in this embodiment, the motion can be made irreversible at predetermined position along the range of travel of plunger 250 through the suitable placement of slots 251 and 252. Finally, FIG. 41 shows handle mechanism 10 at the full stroke position of plunger 250. At this stage, a flange 283 on the plunger grip 280 abuts against button lock 230 and a fastener 211 at the distal end of the handle bottom 212 of handle body 210 engages a bottom notch 282 on the bottom of plunger grip 280 to provide tactile feedback to the user to indicate the full stroke has been achieved. At this stage, plunger 250 is locked and prostatic implant 5 has been fully pushed out of catheter 12 and is ready to be disengaged from either pusher member 13 or pusher head 171, depending on the embodiment of the control release mechanism.

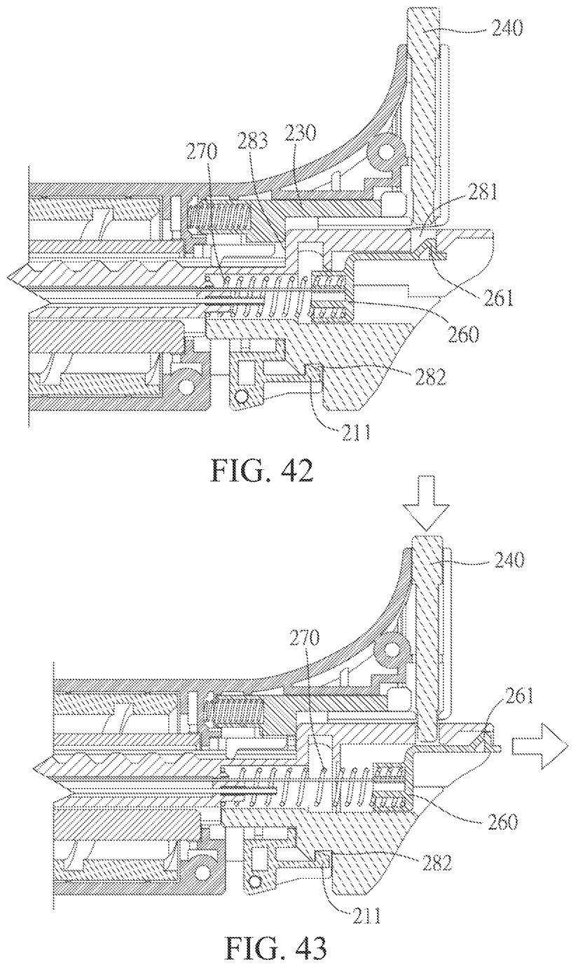

Following deployment, handle mechanism 10 may be employed to disengage prostatic implant 5 by actuating wire latch 15 or another of the control members discussed above. Referring first to FIG. 42, in conjunction with fastener 211 engaging notch 282, flange 283 of plunger cover 280 moves button lock 230 distally to free release button 240. Once release button 240 is not restrained by flange 283, release spring 241 (shown in FIG. 37) drives release button 240 into fastener 261 of actuator 260, disengaging it from perforation 261 as shown in FIG. 43. Consequently, actuator 260 is now automatically driven proximally by actuator spring 270. As discussed above, actuator 260 is coupled to the control member of the control release mechanism, such as wire latch 15, and triggers release of prostatic implant 5.

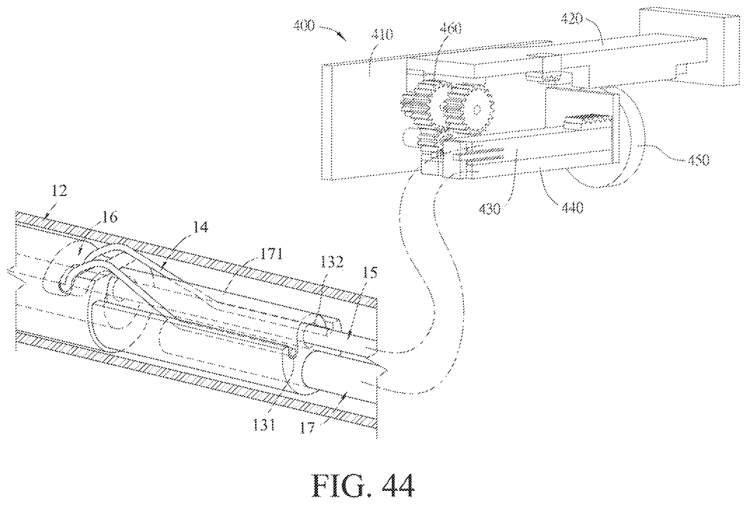

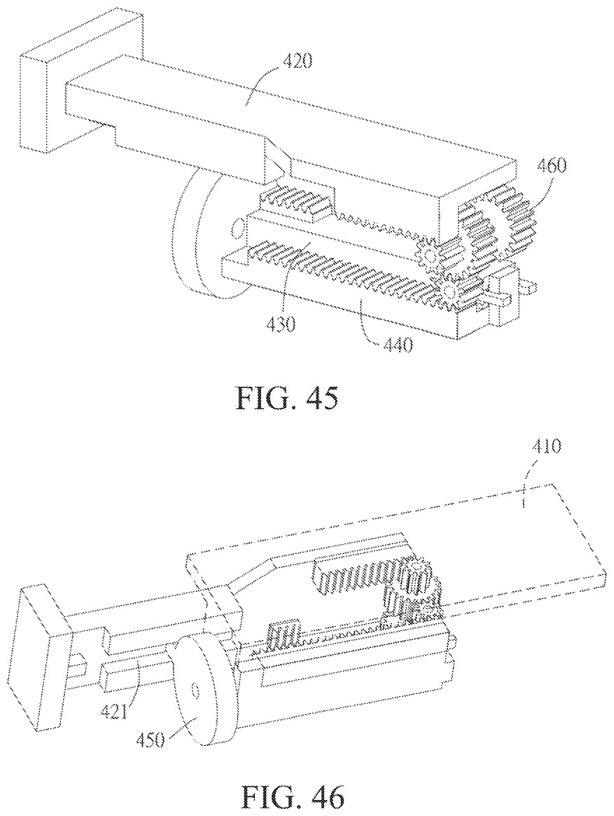

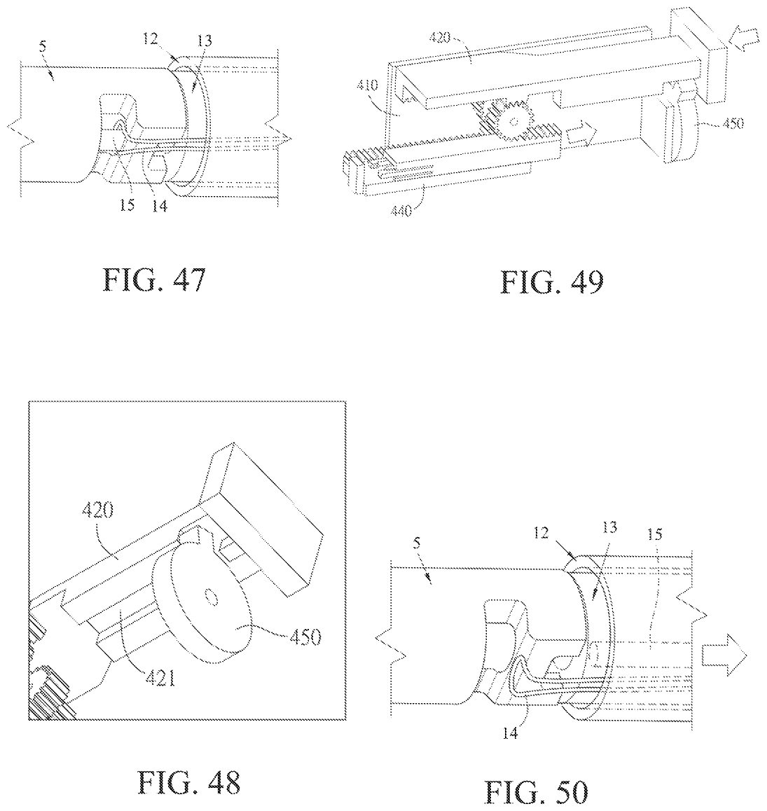

In some embodiments, handle mechanism 10 may employ alternative linkages to facilitate the deployment and/or disengagement stages. For example, FIG. 44 schematically depicts use of an accelerator release set. As shown, this embodiment is presented in the context of use with the control release mechanism discussed above with regard to FIGS. 8-10 but may be adapted as needed to work with the other control members. In particular, a release set 400 may have a support frame 410, a plunger 420, a pusher wire holder 440 coupled with pusher wire 17, a wire latch holder 430 coupled with wire latch 15, a disengage lock 450 and a gear train 460 carried by support frame 410. At the initial stage of deployment, release set 400 has the configuration shown in FIG. 45, with plunger 420 and pusher wire holder 440 coupled through gear train 460 and the track of latch wire holder 430 not coupled in this position. As shown in FIG. 46, disengage lock 450 cannot rotate due to interaction with a slot 421 of plunger 420 and prevents further movement of plunger 420. In this configuration, plunger 420 is pushed to continue deployment of prostatic implant 5 until free of the distal end of catheter 12. As shown in FIG. 47, wire latch 15 still engages the loop of suture 14 to retain prostatic implant 5 at this stage. At this stage, disengage lock 450 can be rotated to allow further travel of plunger 420 as shown in FIG. 48. Accordingly, further travel of plunger 420 now engages gear train 460 with the track of wire latch holder 430 and causes it to be withdrawn as indicated by FIG. 49. As a result, wire latch 15 is withdrawn proximally to free loop of suture 14 that was retained within recess 16 so that prostatic implant is released as shown in FIG. 50.

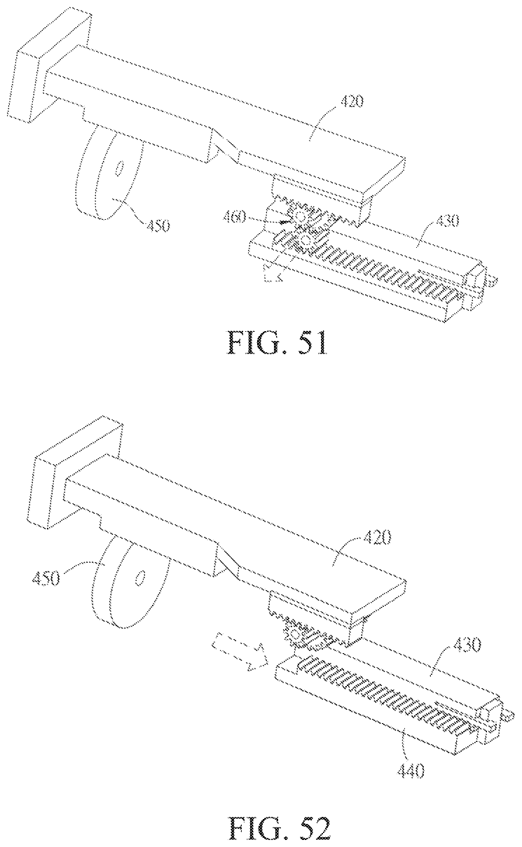

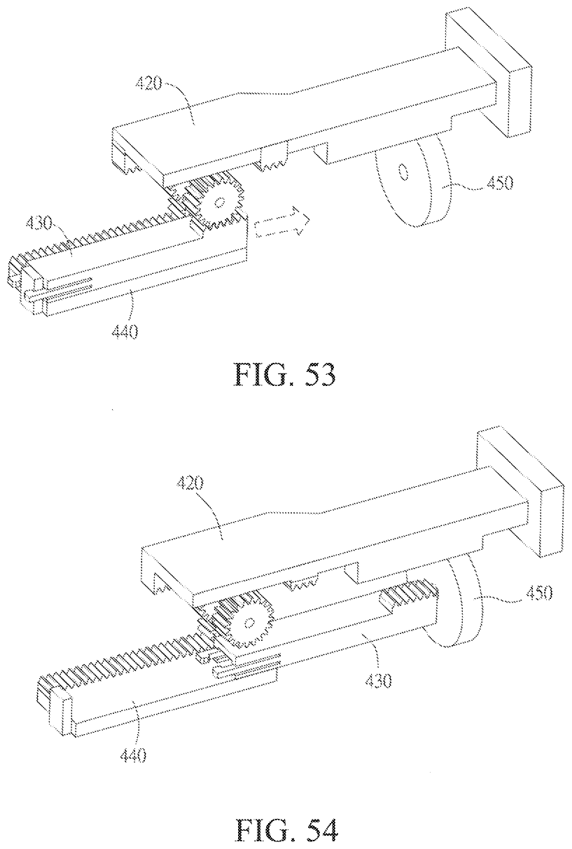

If necessary, a bail-out operation may be performed by pulling out the gears to release gear train 460 as per FIG. 51, advancing pusher wire holder 440 to the distal end as per FIG. 52, drawing back wire latch holder 430 to the proximal end as per FIG. 53 and returning to the original configuration as per FIG. 54.

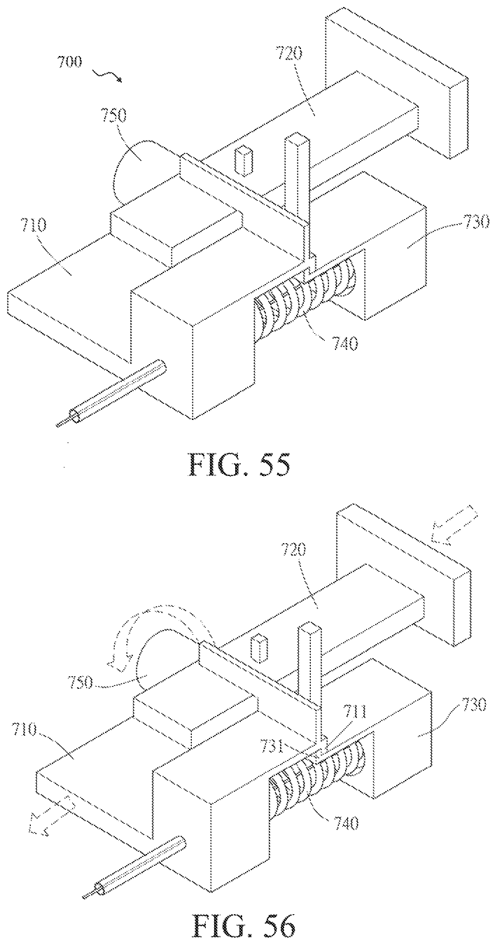

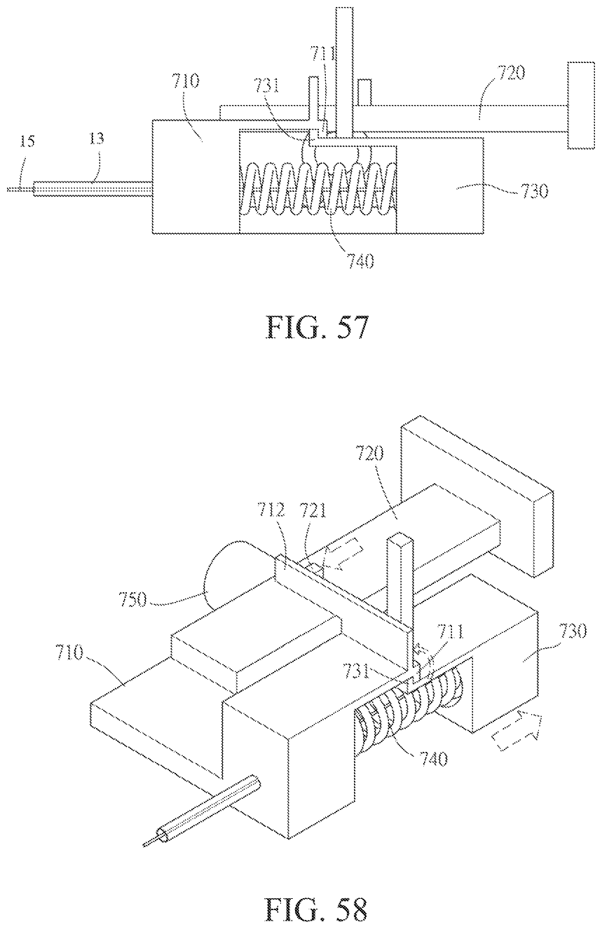

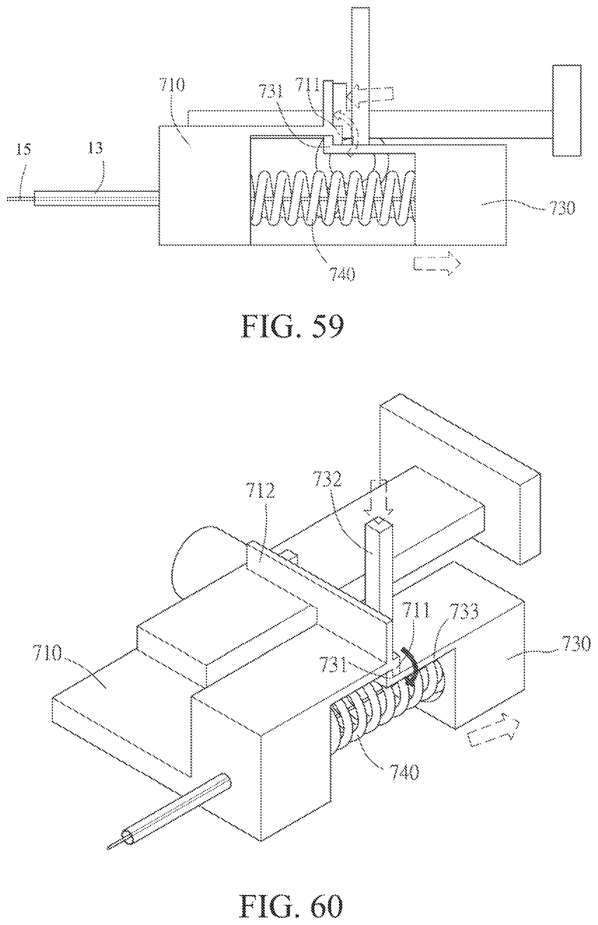

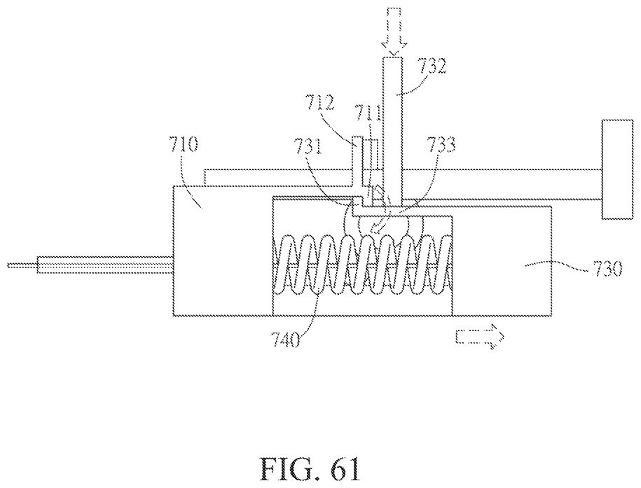

In another embodiment, a release set 700 as shown in FIG. 55 may be used with handle mechanism 10. As shown, release set 700 includes a pusher wire holder 710 coupled with pusher wire 17, a plunger 720, a wire latch holder 730 coupled with wire latch 15, a wire latch spring 740 and a gear train 750. In the deployment stage, plunger 720 is pushed to start the deployment of prostatic implant 5 from the distal end of catheter 12, with pusher wire holder 710 advancing due to engagement of gear train 750 as indicated in FIG. 56. In the side view of FIG. 57, it may be seen that wire latch holder 730 and pusher wire holder 710 move in unison at this stage due to a latch 731 on wire latch holder 730 that engages with a latch 711 on pusher wire holder 710. When the full deployment stage is reached, FIG. 58 shows that a protrusion 721 on the plunger 720 engages and lifts a cantilever beam 712 coupled to latch 711 to cause disengagement from latch 731 as shown in the elevational view of FIG. 58 and the side view of FIG. 59. Once the latches 711, 731 are opened and no longer constrain pusher wire holder 710 and wire latch holder 730 from moving independently, the force from wire latch spring 740 drives wire latch holder 730 proximally to disengage prostatic implant 5. If necessary, a bail-out operation may be performed as shown in the elevational view of FIG. 60 and the side view of FIG. 61, by depressing a bar 732 attached to cantilever beam 733 causing manual deflection and disengagement of latch 731. Again, once the latches 711, 731 are opened and no longer constrain pusher wire holder 710 and wire latch holder 730 from moving independently, the force from wire latch spring 740 drives wire latch holder 730 proximally to disengage prostatic implant 5.

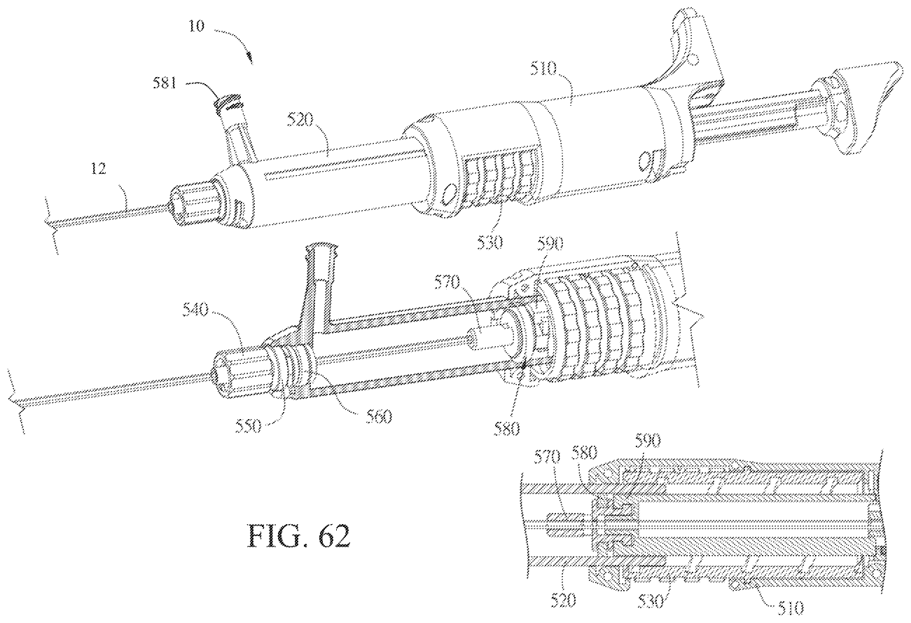

As noted above, use of handle mechanism 10 when delivering and deploying prostatic implant 5 to relieve benign prostatic hyperplasia (BPH) may also involve adjusting the exposed length of catheter 12. Correspondingly, the system can be compatible with cystoscopes having different physical dimensions. Thus, embodiments of this disclosure include handle mechanism 10 as shown in FIG. 62 that includes catheter 12, a handle body 510, a telescopic cylinder 520, a knob rib 530, a Luer lock ring 540, a Luer connector 550, a Luer connector hub 560, a catheter hub 570 to receive catheter 12, a catheter hub seal 580, and a catheter holder 590.

To provide compatibility with different cystoscopes that may have variation in the length of the working channel, the exposed length of catheter 12 may be adjusted by rotating knob rib 530 to extend or retract telescopic cylinder 520. A screw thread 531 formed on an inner surface of knob rib 530 is engaged by one or more projections 521 on an outer surface of telescopic cylinder 520 at the proximal end. Rotation of knob rib 530 correspondingly extends or retracts telescopic cylinder 520 so that a desired length of catheter 12 is exposed. Catheter 12 is connected with catheter hub 570, with catheter hub seal 580 used to seal the space between catheter hub 570 and telescopic cylinder 520 to inhibit liquid leakage from telescopic cylinder 520 into handle body 510. Moreover, another seal ring (not shown) between catheter hub 570 and the catheter holder 590 may seal the space between catheter hub 570 and catheter holder 590. Luer connector seal 551 inhibits liquid leakage from between telescopic cylinder 520 and Luer connector 550.

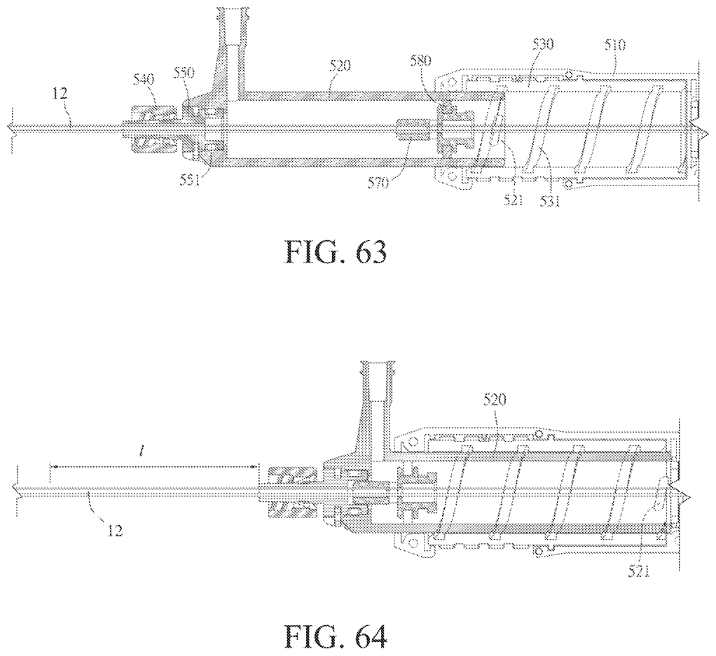

Catheter hub 570 is secured to handle body 510 by catheter holder 590 to prevent relative movement between handle body 510 and catheter 12 during movement of telescopic cylinder 520. Catheter 12 passes through the inner portion of telescopic cylinder 520 via Luer connector 550 and is exposed out of telescopic cylinder 520. It will be appreciated that the exposed portion of catheter 12 will be longer when telescopic cylinder 520 is retracted into handle body 510 and will be correspondingly shorter when telescopic cylinder 520 is extended out of handle body 510. Therefore, through rotation of knob rib 530, the length of exposed catheter is adjustable to facilitate compatibility with different cystoscopes that may have working channels of varying length. For example, FIG. 63 depicts a maximum extension of telescopic cylinder 520 when the one or more projections 521 have reached the most distal position within screw thread 531, such that catheter 12 has a minimum exposed length and FIG. 64 depicts a maximum retraction of telescopic cylinder 520 when the one or more projections 521 have reached the most proximal position within screw thread 531, such that catheter 12 has a maximum exposed length, l.

### Irrigation System

Referring back to FIG. 2, this disclosure also includes irrigation system 11 that may be used in conjunction with the control release 9 and handle 10 mechanisms discussed above for delivering and deploying prostatic implant 5 to relieve benign prostatic hyperplasia (BPH). Irrigation system 11 provides with one or more liquid paths for improving visualization through the cystoscope during the operation. A clearance liquid, such as saline, flows through an inner channel of the catheter and/or through outer diameter lumen formed by an outer surface of the catheter and the inner diameter of a cystoscope's working channel for irrigation. In one aspect, irrigation system 11 provides at least one liquid path through the side arm of handle mechanism 10 such as in FIG. 62.

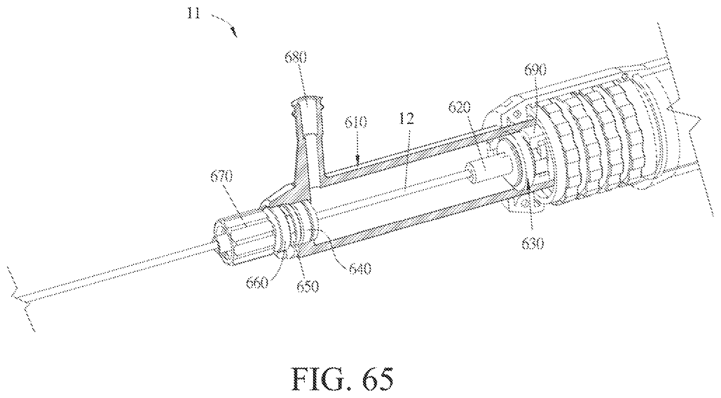

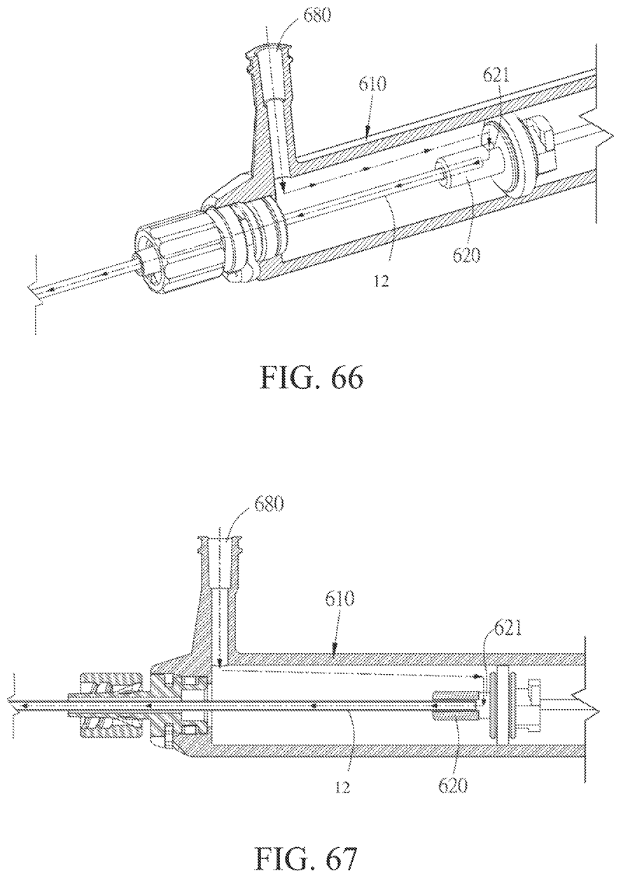

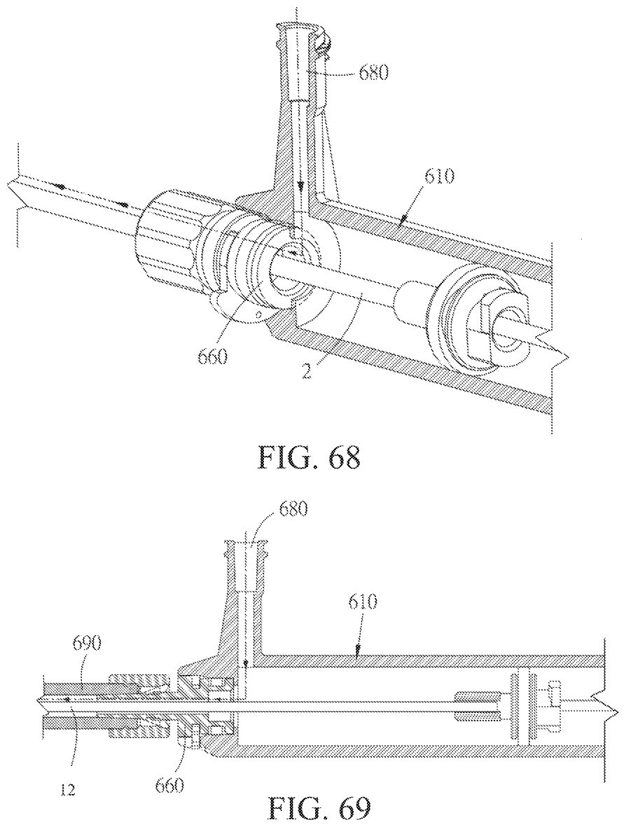

In another embodiment, an irrigation system 11 as shown in FIG. 65 may include catheter 12, a telescopic cylinder 610, a catheter hub 620, a catheter hub seal 630, a Luer connector seal 640, a Luer connector lock 650, a Luer connector 660, a Luer lock ring 670 and a side arm liquid inlet 680. As above, catheter hub seal 630 is used to seal the space between catheter hub 620 and telescopic cylinder 610 to prevent liquid leakage from the telescopic cylinder 610 into the handle body. Moreover, another seal ring (not shown) between catheter hub 620 and catheter holder 690 seals the space between catheter hub 620 and catheter holder 690 to prevent liquid leakage from telescopic cylinder 610 into the handle body. Luer connector seal (640) prevent liquid leakage from the clearance between telescopic cylinder 610 and Luer connector 660. In this embodiment, dual irrigation paths are provided. When saline is introduced into the inner cavity of telescopic cylinder 610 from liquid inlet 680, saline flows along two distinct paths. A first path is defined by saline flow into an inner channel of catheter 12 through an inlet hole 621 on catheter hub 620 and flow out from the opening at the distal end of catheter 12 as indicated by the dot-dash line in the elevational view of FIG. 66 and the side view of FIG. 67. A second path is defined by saline flow from the inner cavity of telescopic cylinder 610 will flow into the outer lumen formed between the outside diameter of catheter 12 and the inside diameter of a cystoscope working channel 690 and out from the opening at the distal end of cystoscope working channel 690 as indicated by the dot-dash line in the elevational view of FIG. 68 and the side view of FIG. 69.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed, and the principles of this disclosure can be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

What is claimed is:

1. A controlled release mechanism for a prostatic implant delivery and deployment system comprising:
a catheter;
a prostatic implant disposed within a distal portion of the catheter,
a pusher member coaxially disposed within the catheter proximal to the prostatic implant;
a disengageable connection between the pusher member and the prostatic implant; and
a control member extending between the disengageable connection and a proximal end of the catheter.

2. The controlled release mechanism of claim 1, wherein the pusher member comprises an elongated tubular member.

3. The controlled release mechanism of claim 1, wherein the pusher member comprises a pusher wire and a pusher head and wherein the disengageable connection is between the prostatic implant and the pusher head.

4. The controlled release mechanism of claim 1, wherein the disengageable connection comprises a wire latch removably disposed in a lumen of the prostatic implant and a suture looping from the pusher member around the wire latch.

5. The controlled release mechanism of claim 4, wherein the pusher member comprises a pusher wire and a pusher head.

6. The controlled release mechanism of claim 5, wherein the suture is secured to the pusher head.

7. The controlled release mechanism of claim 6, wherein the pusher head comprises a distal portion and a proximal portion and wherein the suture is secured to the proximal portion of the pusher head.

8. The controlled release mechanism of claim 5, wherein the suture comprises a continuous loop encircling the pusher wire.

9. The controlled release mechanism of claim 5, wherein the pusher head is conical with a tapered region formed by a plurality of supports.

10. The controlled release mechanism of claim 1, wherein the disengageable connection comprises a fastener at a proximal end of the prostatic implant and a tilt locker secured to the control member and wherein the control member is a pre-shaped wire.

11. The controlled release mechanism of claim 1, wherein the disengageable connection comprises a fastener at a proximal end of the prostatic implant and a chamfer locker secured to the control member.

12. The controlled release mechanism of claim 1, wherein the disengageable connection comprises a fastener at a proximal end of the prostatic implant and a tube anchor secured to the control member.

13. The controlled release mechanism of claim 1, wherein the disengageable connection comprises hooks extending from the pusher member and wherein the hooks are maintained in an outward configuration when supported by the control member.

14. The controlled release mechanism of claim 1, wherein the disengageable connection comprises a lock wire extending into an inner diameter of the prostatic implant having an aperture at a distal end and the control member is a loop routed around a structure of the prostatic implant and through the aperture.

15. The controlled release mechanism of claim 1, wherein the disengageable connection comprises a suture connected to the control member and looped through an aperture formed in a proximal portion of the prostatic implant.

16. A method for deploying and disengaging a prostatic implant comprising:
providing a catheter having a prostatic implant disposed within a distal portion of the catheter, a pusher member coaxially disposed within the catheter proximal to the prostatic implant and a disengageable connection between the pusher member and the prostatic implant;
advancing the pusher member distally until the prostatic implant is beyond a distal end of the catheter;
actuating a control member at a proximal end of the catheter to release the prostatic implant, wherein the control member is coupled to the disengageable connection.

17. The method of claim 16, wherein actuating the control member comprises withdrawing a wire latch from a lumen of the prostatic implant to disengage a suture looping from the pusher member around the wire latch.

18. The method of claim 16, wherein actuating the control member comprises disengaging a lock member associated with the pusher member from a fastener at a proximal end of the prostatic implant.

19. The method of claim 16, wherein hooks extending from the pusher member are maintained in an outward configuration by the control member in a supporting position to engage corresponding notches in a proximal portion of the prostatic implant and wherein actuating the control member comprises withdrawing the control member from the supporting position.

20. The method of claim 16, wherein actuating the control member compresses and withdraws an expandable lock through a proximal neck of the prostatic implant.

21. The method of claim 16, wherein a suture loops from an aperture at a distal end of a lock wire by extending into an inner diameter of the prostatic implant and around a structure of the prostatic implant and wherein actuating the control member comprises withdrawing the suture.

22. The method of claim 16, wherein actuating the control member comprises withdrawing the control member to disengage a suture looped through an aperture formed in a proximal portion of the prostatic implant.

23. The method of claim 22, wherein withdrawing the control member breaks the suture.
